(19) United States
(12) Patent Application Publication
(10) Pub. No.: US 2022/0296155 A1
(43) Pub. Date: Sep. 22, 2022

(54) RESPONSIVENESS TESTING OF A PATIENT HAVING BRAIN STATE CHANGES

(71) Applicant: IVAN OSORIO, Leawood, KS (US)

(72) Inventor: IVAN OSORIO, Leawood, KS (US)

(73) Assignee: FLINT HILLS SCIENTIFIC, L.L.C., LAWRENCE, KS (US)

(21) Appl. No.: 17/832,602

(22) Filed: Jun. 4, 2022

Related U.S. Application Data
(63) Continuation of application No. 12/756,065, filed on Apr. 7, 2010.

(51) Int. Cl. A61B 5/369; A61B 5/16; A61B 5/00; G16H 50/30; G16H 40/63

(52) U.S. Cl. CPC A61B 5/369; A61B 5/165; A61B 5/7264; A61B 5/4094; A61B 5/7275; G16H 50/30; G16H 40/63; A61B 5/4035; G16H 20/70

(57) ABSTRACT

A method of determining a responsiveness of a patient having brain state changes, comprising receiving an indication of a triggering event; administering to the patient, in response to the indication, a test of responsiveness; determining, based upon a result of the test, at least one responsiveness parameter selected from the group consisting of (i) a time of occurrence of a change in the patient's responsiveness, (ii) a duration of a change in the patient's responsiveness; (iii) a magnitude of a change in the patient's responsiveness, (iv) a time interval from the indication of event occurrence to a change in the patient's responsiveness, (v) a type of change in the patient's responsiveness, (vi) an estimation of a seizure severity; (vii) a classification of a seizure into clinical or subclinical; (viii) a classification of a clinical seizure into simple partial, complex partial, or generalized; (ix) an assessment of efficacy of a therapy for the patient's medical condition; (x) an assessment of the state of the disease and formulation of a prognosis for the patient; (xi) an estimation of a risk of injury or death for the patient; and (xii) two or more thereof. A medical device system capable of implementing the method.

### CROSS-REFERENCE TO RELATED APPLICATONS

This presently being filed application is a continuation of and claims priority to co-pending U.S. patent application Ser. No. 12/756,065 entitled “Responsiveness Testing of a Patient Having Brain State Changes”, filed on Apr. 7, 2010, which is incorporated by reference in its entirety.

### FIELD OF THE INVENTION

This invention relates generally to medical device systems and, more particularly, to medical device systems capable of testing the responsiveness of a patient having brain state changes. Testing of responsiveness of a patient may be used to determine a time at which loss of function for the patient occurs.

### DESCRIPTION OF THE RELATED ART

Therapies using electrical currents or fields to provide a therapy to a patient (electrotherapy) are beneficial for certain neurological disorders, such as epilepsy. Implantable medical devices have been effectively used to deliver therapeutic electrical stimulation to various portions of the human body (e.g., the vagus nerve) for treating various medical conditions, including epilepsy. As used herein, “stimulation,” “neurostimulation,” “stimulation signal,” or “neurostimulation signal” refers to the application of an electrical, mechanical, magnetic, electro-magnetic, photonic, acoustic, physiological, cognitive, and/or chemical signal to a neural structure in the patient's body. The signal is an exogenous signal that is distinct from the endogenous electro-chemical activity inherent to the patient's body and the environment. In other words, the stimulation signal (whether electrical, mechanical, magnetic, electro-magnetic, photonic, acoustic or chemical in nature) applied to a cranial nerve or to other nervous tissue structure in the present invention is a signal applied from a medical device, e.g., a neurostimulator.

A “therapeutic signal” refers to a stimulation signal delivered to a patient's body with the intent of treating a medical condition through a suppressing (blocking) or modulating effect to neural tissue. The effect of a stimulation signal on neuronal activity may be inhibitory (suppressing) or excitatory (expressing); additionally, the effect may be immediate (“all or none”) or the result of spatio-temporal summation of stimuli (modulation or biasing), a process that lacks the immediacy associated with “all or none” responses. However, for simplicity, the terms “stimulation” and “modulation,” and variants thereof, are used interchangeably herein. In general, however, the delivery of an exogenous signal itself refers to “stimulation” of the neural structure, while the effects of that signal, if any, on the electrical activity of the neural structure are properly referred to as “modulation,” which may manifest as either inhibition (suppression) or excitation (expression). Furthermore, depending upon myriad factors such as the history (recent and distant) of the nervous system, stimulation parameters and time of day, to name a few, the effects of stimulation (with the same parameters) upon the neural tissue may be excitatory or inhibitory, facilitatory or disfacilitatory and may suppress, enhance or leave unaltered, the neuronal activity it intends to control. In spite of these vagaries, there is evidence of a suppressing effect of a stimulation signal on abnormal neural tissue activity, specifically of epileptic seizures (see Osorio et al., Ann Neurol 2005; Osorio & Frei IJNS 2009). Suppression of abnormal neural activity is a threshold or suprathreshold process and the temporal scale over which it occurs is usually in the order of a few milliseconds to hundreds of milliseconds. Modulation of abnormal or undesirable neural activity, unlike suppression is a “sub-threshold” process in the spatio-temporal domain that may summate and result under certain conditions, in threshold or suprathreshold neural events. The temporal scale of modulation is much longer than that associated with “all or none” responses. Wave annihilation or reduction through collision with identical, similar or dissimilar waves, or by “pushing” them (the waves) into their “null space” or “black hole” (Winfree; Osorio & Frei 2009) are techniques that rely on stimulation but for which concepts of inhibition or excitation as conventionally used in electrophysiology may not apply. These forms of annihilation (via collision and phase resetting) fall within the purview of wave mechanics and topology. Those skilled in the art realize that there are multiple approaches (and mechanisms) for controlling undesirable oscillations via stimulation (see Osorio et al, Ann Neurol 2005; Kalitzin et al.; Sunderam et al).

In some embodiments, electrotherapy may be provided by implanting an electrical device, i.e., an implantable medical device (IMD), inside a patient's body stimulation of a nervous tissue, such as a cranial nerve. Generally, electrotherapy signals that perform neuromodulation are delivered by the IMD via one or more leads or wirelessly. When applicable, the leads generally terminate at their distal ends in one or more electrodes, and the electrodes, in turn, are coupled to tissue in the patient's body. For example, a number of electrodes may be attached to various points of a nerve or other tissue inside a human body for delivery of a neurostimulation signal.

While contingent (also referred to as “closed-loop,” “active,” or “feedback” stimulation (i.e., electrotherapy applied in response to sensed information, such as heart rate) stimulation schemes have been proposed, conventional vagus nerve stimulation (VNS) is non-contingent, programmed periodic stimulation. Specifically, conventional vagus nerve stimulation usually involves a series of grouped electrical pulses defined by an “on-time” (such as 30 sec) and an “off-time” (such as 5 min). This type of stimulation is also referred to as “open-loop,” “passive,” or “non-feedback” stimulation. Each sequence of pulses during an on-time may be referred to as a “pulse burst.” The burst is followed by the off-time period in which no signals are applied to the nerve. During the on-time, electrical pulses of a defined electrical current (e.g., 0.5-3.5 milliamps) and pulse width (e.g., 0.25-1.0 milliseconds) are delivered at a defined frequency (e.g., 20-30 Hz) for a certain duration (e.g., 10-60 seconds). The on-time and off-time parameters together define a duty cycle, which is the ratio of the on-time to the combination of the on-time and off-time, and which describes the percentage of time that the electrical signal is applied to the nerve.

In conventional VNS, the on-time and off-time may be programmed to define an intermittent pattern in which a repeating series of electrical pulse bursts are generated and applied to a cranial nerve such as the vagus nerve. The off-time is provided to minimize adverse effects and conserve power. If the off-time is set at zero, the electrical signal in conventional VNS may provide continuous stimulation to the vagus nerve. Alternatively, the off time may be as long as one day or more, in which case the pulse bursts are provided only once per day or at even longer intervals. Typically, however, the ratio of “off-time” to “on-time” may range from about 0.5 to about 10.

In addition to the on-time and off-time, the other parameters defining the electrical signal in conventional VNS may be programmed over a range of values. The pulse width for the pulses in a pulse burst of conventional VNS may be set to a value not greater than about 1 msec, such as about 250-500 μsec, and the number of pulses in a pulse burst is typically set by programming a frequency in a range of about 20-150 Hz (i.e., 20 pulses per second to 150 pulses per second). A non-uniform frequency may also be used. Frequency may be altered during a pulse burst by either a frequency sweep from a low frequency to a high frequency, or vice versa. Alternatively, the timing between adjacent individual signals within a burst may be randomly changed such that two adjacent signals may be generated at any frequency within a range of frequencies.

Of the approximately 60 million people worldwide affected with epilepsy, roughly 23 million people suffer from epilepsy resistant to multiple medications (Kwan et al. 2000). In the USA alone, the annual cost of epilepsy care is USD 12 billion (in 1995 dollars), most of which is attributable to subjects with pharmaco-resistant seizures (Begley et al. 2000). Pharmaco-resistant seizures are associated with an increase in mortality and morbidity (compared to the general population and to epileptics whose seizures are controlled by medications) and with markedly degraded quality of life for patients. Seizures may impair motor control, responsiveness to a wide class of stimuli, and other cognitive functions. The sudden onset of a patient's impairment of motor control, responsiveness, and other cognitive functions precludes the performance of necessary and even simple daily life tasks such as driving a vehicle, cooking, or operating machinery, as well as more complex tasks such as acquiring knowledge and socializing.

The deleterious impacts of epilepsy on patients' health and well-being are compounded by the inability to gather, among others, accurate information about event frequency and severity. Event diaries (generated by the patient and/or caregivers) are utterly inadequate (Blum, 1996; Elger 2007) in that event counts/frequencies are grossly underestimated and severity is not measurable due to: a) lack of useful, representative metrics and b) the inability of even experts in the field (epileptologists) to precisely and objectively quantify them based on visual observation. Automated means for quantification of event frequency and severity would allow the stratification of patients by severity, estimation of risks injury and death, formulation of prognosis, tracking the progression of the disorder, and objective assessment of therapeutic efficacy, without which advances are in this field are unlikely to occur or are meager. However, to our knowledge, no practical automated means for quantification of event frequency and severity using signals different from brain electrical signals are publicly available as of this writing, let alone any suitable for rigorous and valid assessment of therapeutic efficacy.

### SUMMARY OF THE INVENTION

In one aspect of the present invention, a method for determining responsiveness of a patient having brain state changes is provided. The method comprises receiving an indication of the occurrence of a triggering event; administering to the patient, in response to the indication, a test of responsiveness; and determining, based upon a result of the test, at least one responsiveness parameter selected from the group consisting of (i) a time of occurrence of a change in the patient's responsiveness, (ii) a duration of a change in the patient's responsiveness; (iii) a magnitude of a change in the patient's responsiveness, (iv) a time interval from the indication of event occurrence to a change in the patient's responsiveness, (v) a type of change in the patient's responsiveness, (vi) an estimation of a seizure severity; (vii) a classification of a seizure into clinical or subclinical; (viii) a classification of a clinical seizure into simple partial, complex partial, or generalized; (ix) an assessment of efficacy of a therapy for the patient's medical condition; (x) an assessment of the state of the disease and formulation of a prognosis for the patient; (xi) an estimation of a risk of injury or death for the patient; and (xii) two or more thereof.

In another aspect of the present invention, a computer readable program storage unit encoded with instructions that, when executed by a computer, perform the method discussed above.

In another aspect of the present invention, a medical device system for determining a responsiveness of a patient having brain state changes, comprising a receiving unit adapted to receive an indication of a triggering event; a responsiveness testing unit adapted to administer a test of responsiveness to a patient in response to the indication; a determination unit adapted to receive a result of the test of responsiveness from the responsiveness testing unit and to make at least one determination selected from the group consisting of (i) a time of occurrence of a change in the patient's responsiveness, (ii) a duration of a change in the patient's responsiveness; (iii) a magnitude of a change in the patient's responsiveness, (iv) a time interval from the indication of event occurrence to a change in the patient's responsiveness, (v) a type of change in the patient's responsiveness, (vi) an estimation of a seizure severity; (vii) a classification of a seizure into clinical or subclinical; (viii) a classification of a clinical seizure into simple partial, complex partial, or generalized; (ix) an assessment of efficacy of a therapy for the patient's medical condition; (x) an assessment of the state of the disease and formulation of a prognosis for the patient; (xi) an estimation of a risk of injury or death for the patient; and (xii) two or more thereof.

The impairment of motor function or of responsiveness that characterizes certain types of events, are associated with high risk for serious injuries, even death, and of inappropriate behavior that further isolates the patient socially. However, since in certain types of events these impairments lag behind the onset of abnormal electrical brain activity, a “natural” window exists during which intervention would minimize these risks. Automated warning of impending impairment of motor function or of responsiveness would minimize risk of injury and social embarrassment, particularly in the case of certain complex partial and secondarily generalized events, as well as allow patients to safely perform certain activities precluded by this disorder.

### BRIEF DESCRIPTION OF THE DRAWINGS

The invention may be understood by reference to the following description taken in conjunction with the accompanying drawings, in which like reference numerals identify like elements, and in which:

FIG. 1A provides a stylized diagram of an implantable medical device implanted into a patient's body for providing a therapeutic electrical signal to a neural structure of the patient's body, in accordance with one illustrative embodiment of the present invention;

FIG. 1B provides a stylized diagram of a medical device system comprising an implantable medical device implanted into a patient's body for providing a therapeutic electrical signal to a neural structure of the patient's body, and an external device for administering a responsiveness test to a patient, in accordance with another illustrative embodiment of the present invention;

FIG. 2A is a block diagram of a medical device system that includes a medical device, in accordance with one illustrative embodiment of the present invention;

FIG. 2B is a block diagram of a medical device system that includes a medical device and a responsiveness test unit, in accordance with one illustrative embodiment of the present invention;

FIG. 3A is a block diagram of a medical device system that includes a medical device, in accordance with one illustrative embodiment of the present invention;

FIG. 3B is a block diagram of a medical device system that includes a medical device and a responsiveness test unit, in accordance with one illustrative embodiment of the present invention;

FIG. 4A is a block diagram of a medical device system that includes a medical device, in accordance with one illustrative embodiment of the present invention;

FIG. 4B is a block diagram of a medical device system that includes a medical device and a responsiveness test unit, in accordance with one illustrative embodiment of the present invention;

FIG. 5 illustrates a block diagram of a change in autonomic or neurologic index detection unit of the medical device system, in accordance with one illustrative embodiment of the present invention;

FIG. 6A illustrates a flowchart depiction of determining a responsiveness of a patient, in accordance with one illustrative embodiment of the present invention;

FIG. 6B illustrates a flowchart depiction of determining a responsiveness of a patient in combination with the delivery and/or modification of therapy, in accordance with one illustrative embodiment of the present invention;

FIG. 6C illustrates a flowchart depiction of determining a responsiveness of a patient in combination with an override based on an autonomic or neurologic index value, in accordance with one illustrative embodiment of the present invention;

FIG. 6D depicts one particular embodiment of administering a test of the patient's responsiveness, in accordance with one illustrative embodiment of the present invention; and

FIG. 6E depicts one particular embodiment of administering a test of the patient's responsiveness, in accordance with one illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

This document does not intend to distinguish between components that differ in name but not function. In the following discussion and in the claims, the terms “including” and “includes” are used in an open-ended fashion, and thus should be interpreted to mean “including, but not limited to.” Also, the term “couple” or “couples” is intended to mean either a direct or an indirect electrical connection. “Direct contact,” “direct attachment,” or providing a “direct coupling” indicates that a surface of a first element contacts the surface of a second element with no substantial attenuating medium there between. The presence of small quantities of substances, such as bodily fluids, that do not substantially attenuate electrical connections does not vitiate direct contact. The word “or” is used in the inclusive sense (i.e., “and/or”) unless a specific use to the contrary is explicitly stated.

The term “electrode” or “electrodes” described herein may refer to one or more stimulation electrodes (i.e., electrodes for delivering an electrical signal generated by an IMD to a tissue), sensing electrodes (i.e., electrodes for sensing a physiological indication of a patient's body), and/or electrodes that are capable of delivering a stimulation signal, as well as performing a sensing function.

Cranial nerve stimulation has been proposed to treat a number of medical conditions pertaining to or mediated by one or more structures of the nervous system of the body, including epilepsy and other movement disorders, depression, anxiety disorders and other neuropsychiatric disorders, dementia, traumatic brain injury, coma, migraine headache, obesity, eating disorders, sleep disorders, cardiovascular disorders (such as congestive heart failure and atrial fibrillation), hypertension, endocrine disorders (such as diabetes and hypoglycemia), and pain (including neuropathic pain and fibromyalgia), among others. See, e.g., U.S. Pat. Nos. 4,867,164; 5,299,569; 5,269,303; 5,571,150; 5,215,086; 5,188,104; 5,263,480; 6,587,719; 6,609,025; 5,335,657; 6,622,041; 5,916,239; 5,707,400; 5,231,988; 5,330,515; 6,961,618; 7,457,665; and 7,630,757; among others. Despite the numerous disorders for which cranial nerve stimulation has been proposed or suggested as a treatment option, the mechanisms of action of stimulation for many (if not all) cranial nerves remain relatively poorly understood.

There is a wide range of medical disorders for which VNS may be prescribed. Among these, those manifesting with events characterized by sudden loss of consciousness (and inevitably of postural tone) or of responsiveness/awareness (without loss of consciousness and of postural tone) are particularly hazardous and disabling to those who suffer from them. Loss of consciousness results invariably in falls to the ground which may be associated with serious bodily and brain injuries; loss of responsiveness (during which patients remain awake but lack discernment) are often the cause of serious vehicular and household accidents (such as fires, burns). Sudden loss of consciousness may be cardiovascular/autonomic or neurological in nature; loss of awareness is almost always neurological in nature. Among the neurological causes of loss of consciousness (with or without abnormal motor activity) epileptic seizures rank second to cardiovascular/autonomic dysfunction; for loss of responsiveness, epileptic seizures rank highest. Epileptic seizures are characterized by sudden, transient increases (above the normal level) in neuronal membrane voltages, commonly associated with changes in autonomic function. Seizures may affect all bodily functions under autonomic control, most notably cardio-respiratory and also temperature (Sunderam & Osorio), pupillary, skin resistance control, sphincter tone, peristalsis, etc. By identifying which bodily functions are affected by an epileptic seizure, changes in the signals or indices associated with the affected function(s) may be used to detect seizures automatically. Specifically seizures may be detected via: a) brain electrical signals recorded from the scalp (electroencephalogram; EEG) or directly from the brain (electrocorticogram; ECoG); b) autonomic signals or indices such as changes in heart and respiratory activity rate, pupillary size. For example, increases in heart rates (tachychardia) and respiratory rates (tachypnea or hyperventilation) often occur in patients with partial seizures while they are motionless. The occurrence of autonomic changes during seizures is consistent with the fact that autonomic functions are under the control of the brain (central nervous system) (Brodal) which is the site of epileptogenesis and ictiogenesis. Autonomic or neurologic index or indices is/are used herein to refer to any detectable state or change of state reflective of the function of one or more aspects of the autonomic nervous system of the patient.

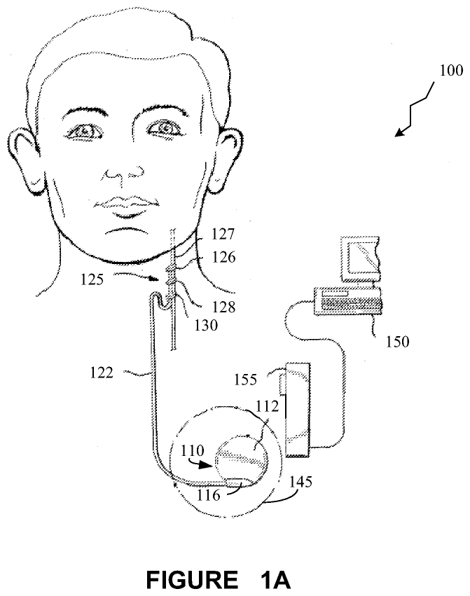

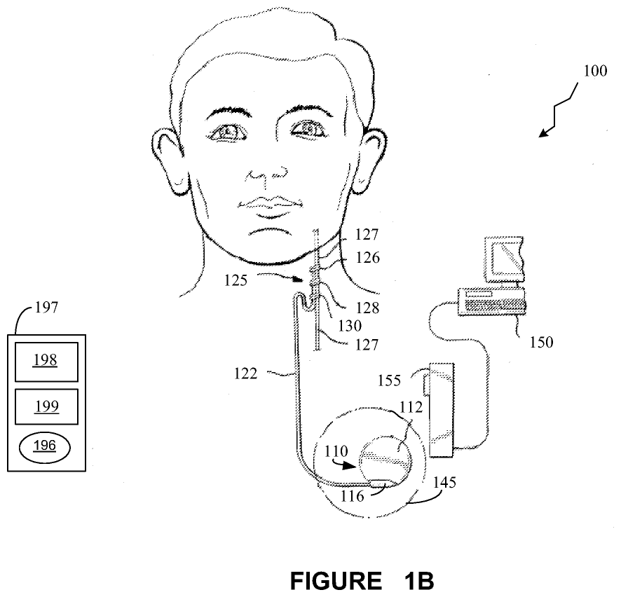

Although not so limited, a system capable of implementing embodiments of the present invention is described below. FIG. 1A depicts a stylized implantable medical system (IMD) 100 for implementing one or more embodiments of the present invention. An electrical signal generator 110 is provided, having a main body 112 comprising a case or shell with a header 116 for connecting to an insulated, electrically conductive lead assembly 122. The generator 110 is implanted in the patient's chest in a pocket or cavity formed by the implanting surgeon just below the skin (indicated by a dotted line 145), similar to the implantation procedure for a cardiac pacemaker pulse generator.

A nerve electrode assembly 125, preferably comprising a plurality of electrodes having at least an electrode pair, is conductively connected to the distal end of the lead assembly 122, which preferably comprises a plurality of lead wires (one wire for each electrode). Each electrode in the electrode assembly 125 may operate independently or alternatively, may operate in conjunction with the other electrodes. In one embodiment, the electrode assembly 125 comprises at least a cathode and an anode. In another embodiment, the electrode assembly comprises one or more unipolar electrodes.

Lead assembly 122 is attached at its proximal end to connectors on the header 116 of generator 110. The electrode assembly 125 may be surgically coupled to the vagus nerve 127 in the patient's neck or at another location, e.g., near the patient's diaphragm or at the esophagus/stomach junction. Other (or additional) cranial nerves such as the trigeminal and/or glossopharyngeal nerves may also be used to deliver the electrical signal in particular alternative embodiments. In one embodiment, the electrode assembly 125 comprises a bipolar stimulating electrode pair 126, 128 (i.e., a cathode and an anode). Suitable electrode assemblies are available from Cyberonics, Inc., Houston, Tex., USA as the Model 302 electrode assembly. However, persons of skill in the art will appreciate that many electrode designs could be used in the present invention. In one embodiment, the two electrodes are wrapped about the vagus nerve, and the electrode assembly 125 may be secured to the vagus nerve 127 by a spiral anchoring tether 130 such as that disclosed in U.S. Pat. No. 4,979,511 issued Dec. 25, 1990 to Reese S. Terry, Jr. and assigned to the same assignee as the instant application. Lead assembly 122 may be secured, while retaining the ability to flex with movement of the chest and neck, by a suture connection to nearby tissue (not shown).

In some embodiments, the electrode assembly 125 may comprise temperature sensing elements and/or heart beat sensor elements for detection of electrical, mechanical or acoustic activity. Other sensors for other autonomic indices may also be employed. Both closed-loop and open-loop stimulation may be combined or delivered by a single IMD according to the present invention. Either or both modes may be appropriate to treat a specific patient under observation.

The electrical pulse generator 110 may be programmed with an external device (ED) such as computer 150 using programming software known in the art. A programming wand 155 may be coupled to the computer 150 as part of the ED to facilitate wireless radio frequency (RF) communication between the computer 150 and the implanted pulse generator 110. The programming wand 155 and computer 150 permit non-invasive communication with the generator 110 after the latter is implanted. In systems where the computer 150 uses one or more channels in the Medical Implant Communications Service (MICS) bandwidths, the programming wand 155 may be omitted to permit more convenient communication directly between the computer 150 and the pulse generator 110.

Turning now to FIG. 1B, the depicted embodiment shows, in addition to the IMD 100, a responsiveness testing input/output (I/O) unit 197 for implementing one or more embodiments of the present invention. The responsiveness testing I/O unit 197 contains an input device 199, an output device 198, and a control and communication unit 196.

The output device 198 is configured to present an output to the patient when it receives instructions and/or commands to do so from the control and communication unit 196. By “output” is meant a visual, auditory, tactile, olfactory or gustatory stimulus or signal perceptible by one or more of the senses of a patient. Exemplary output devices 198 include, but are not limited to, visual devices, such as LCD, LED, or other displays, which may output a light, graphics, text, animation, or video, among others, or two or more thereof; audio devices, such as speakers, which may output sound, synthesized speech, recorded speech, or live speech, among others, or two or more thereof; and tactile devices, which may output

Braille text, vibration, heat, or cold, among others, or two or more thereof The output device 198 may also comprise two or more of the devices described above, among others; for example, the output device 198 may comprise a visual device and an audio device, for example, an LCD screen and a speaker, among others. In various embodiments, the output device 198 may be housed in the medical device 200 or an external unit 270.

The input device 199 is configured to receive an input from the patient when it receives instructions to do so from the control and communication unit 196. By “input” is meant any state or change in state of the device effected by one or more actions of the patient.

Exemplary input devices 199 include, but are not limited to, touchscreens, buttons, switches, microphones, and cameras, among others, or two or more thereof. In various embodiments, the input device 199 may be housed in the medical device 200 or an external unit 270.

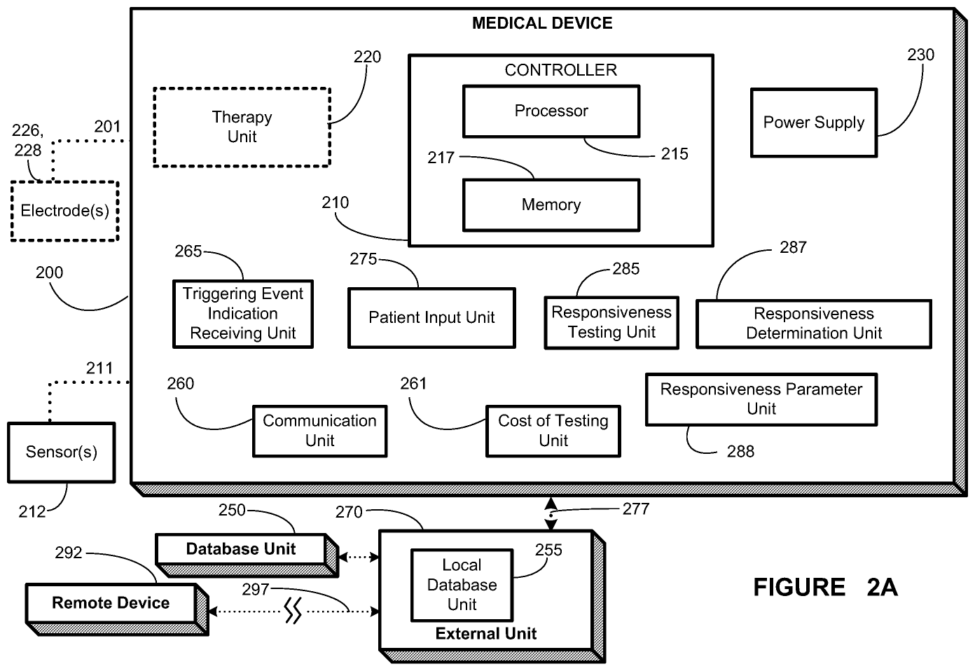

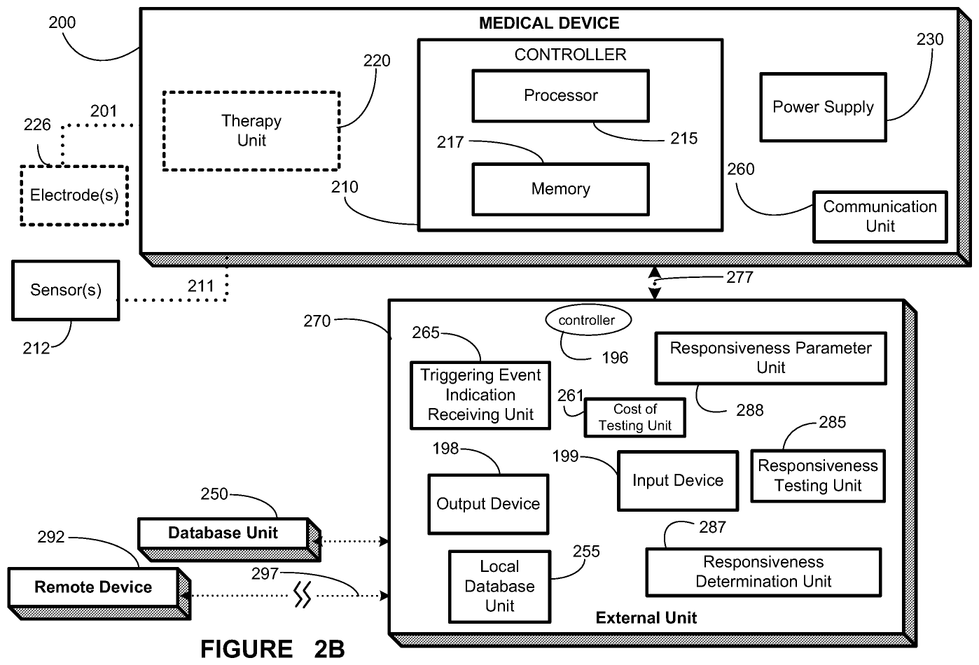

The control and communication unit 196, as mentioned, provides instructions to the output device 198 to present an output and to the input device 199 to receive an input. It also provides instructions for the functions of a triggering event indication receiving unit (265, FIG. 2B), a responsiveness testing unit (285, FIG. 2B), a responsiveness determination unit (287, FIG. 2B), and a responsiveness parameter unit (288, FIG. 2B), which functions each involve the receipt of, transmission of, and internal handling of, data, as will be discussed in more detail with reference to FIG. 2B, below.

The responsiveness testing I/O unit 197 is shown as a single discrete unit in FIG. 1B, but other embodiments are possible. The responsiveness testing I/O unit 197 may be external to the patient's body, and in a further embodiment, may be configured to be held in the hand. As should be apparent, in other embodiments, the responsiveness testing I/O unit 197 may not be configured to be held in the hand, but may instead be placed elsewhere on the patient's body (e.g., on the wrist, among other locations), on a table, desk, nightstand, floor, or on, in, or otherwise sited with reference to a feature of the patient's environment. The hardware, the software, or both of the responsiveness testing I/O unit 197 may be especially designed for responsiveness testing, but need not be; for example, in one embodiment, the responsiveness testing I/O unit 197 is embodied as software in a cellular telephone, a smartphone (e.g., an Apple iPhone® or a smartphone sold by BlackBerry, Palm, Motorola, HTC, or the like), a personal digital assistant (PDA), or another programmable handheld electronic device (e.g., an Apple iPod®). In another embodiment, the responsiveness testing I/O unit 197 is embodied as software on a netbook, notebook, or desktop computer, such as one running an operating system such as Microsoft Windows, Apple Macintosh OS X, or Linux, among others. In some embodiments, the patient may be provided with a plurality of responsiveness testing I/O units 197, networked together in a system capable of receiving inputs and providing outputs to any of the units 197 comprising the network. For certain applications the responsiveness testing I/O unit 197 may be implanted into the patient's body.

Also, in various embodiments, portions of the responsiveness testing I/O unit 197 may be housed in separate units. For example, the output device 198 may be a monitor or a speaker of a computer, and the input device 199 may be a touchscreen, button, switch, microphone, or camera embodied in a handheld device. For another example, the output device 198 may be a monitor or a speaker of a handheld device, and the input device 199 may be a keyboard, mouse, microphone, or camera of a computer. For still another example, the output device 198 may be a monitor or a speaker of a handheld device, and the input device 199 may be a magnetic swipe sensor or a tap sensor of the IMD 100.

Also, although FIG. 1B shows the responsiveness testing I/O unit 197 in proximity to the IMD 100, as should be apparent, the responsiveness testing I/O unit 197 may be used entirely separately from the IMD 100. For example, a patient having brain state changes may use the responsiveness testing I/O unit 197 without having the IMD 100 implanted within his or her body. In such embodiments, the invention may lack any implanted component. Thus, IMD 100 may be absent and the invention may comprise responsiveness testing I/O unit 197 alone or with an external medical device.

Turning now to FIG. 2A, a block diagram depiction of the medical device (MD) 200 is provided, in accordance with one illustrative embodiment of the present invention. The MD 200 (such as implantable generator 110 from FIG. 1) may comprise a controller 210 capable of controlling various aspects of the operation of the MD 200. In some embodiments, the controller 210 is capable of receiving data and causing a therapy unit 220 to generate and deliver a therapy, such as an electrical signal to target tissues of the patient's body for treating a medical condition using, for example, electrodes 226, 228. Therapy unit 220 is optional, as indicated by the dotted line. In some embodiments, therapy unit 220 is absent. For example, the controller 210 may receive instructions from another device, or may cause the electrical signal to be generated and delivered based on calculations and programming internal to MD 200. The controller 210 is capable of affecting substantially all functions of the MD 200. The MD 200 may be an external device, or in an alternative embodiment, an implantable medical device.

The controller 210 may comprise various components, such as a processor 215, a memory 217, etc. The processor 215 may comprise one or more microcontrollers, microprocessors, etc., capable of performing various executions of software components. The memory 217 may comprise various memory portions where a number of types of data (e.g., internal data, external data instructions, software codes, status data, diagnostic data, etc.) may be stored. The memory 217 may comprise one or more of random access memory (RAM), dynamic random access memory (DRAM), electrically erasable programmable read-only memory (EEPROM), flash memory, volatile memory, non-volatile memory, etc.

In one embodiment, MD 200 may be an implantable medical device, and coupling 201 may comprise a lead assembly such as lead assembly 122 (FIG. 1). In another embodiment, MD 200 may be external to the patient's body, and may be coupled to an implanted lead via a wireless or inductive coupling, such as an RF inductive coupling. In a still further embodiment, MD 200 may be external to the patient's body and electrodes 226, 228 may also be external to the patient's body. Whether MD 200 is an implantable or external unit, a therapeutic electrical signal may be delivered to the electrodes 226, 228 by the therapy unit 220 based upon instructions from the controller 210. The therapy unit 220 may comprise various circuitry, such as electrical signal generators, impedance control circuitry to control the impedance “seen” by the leads, and other circuitry that receives instructions relating to the delivery of the electrical signal to tissue. Therapy unit 220 may be configured to deliver biphasic, charge balanced pulses, multiphasic pulses, or monophasic pulses. Therapy unit 220 may deliver constant current or constant voltage. Further, therapy unit 220 may be configured to deliver magnetic currents, or to operate as a drug administration and/or thermal control unit.

In other embodiments, coupling 201 is operatively coupled to an electrode, wherein the electrode is adapted to be coupled to at least one of a portion of a brain structure of the patient, a cranial nerve of a patient, an organ for special senses of a patient, the spinal cord of a patient, a spinal cord root of a patient, a sympathetic nerve structure of the patient, a peripheral nerve of the patient, the skin of the patient, or a muscle of the patient.

In some embodiments, therapy unit 220 as well as coupling 201 and electrodes 226, 228 may be omitted. In other words, the responsiveness testing described herein can be performed by MD 200 whether or not a therapy for the patient's medical is provided.

The MD 200 may also comprise a power supply 230. The power supply 230 may comprise a battery, voltage regulators, capacitors, etc., to provide power for the operation of the MD 200, including delivering the therapeutic electrical signal. The power supply 230 comprises a power source that in some embodiments may be rechargeable. In other embodiments, a non-rechargeable power source may be used. The power supply 230 provides power for the operation of the MD 200, including electronic operations and the electrical signal generation and delivery functions. The power supply 230 may comprise a lithium/thionyl chloride cell or a lithium/carbon monofluoride (LiCFx) cell for implantable embodiments, and more common watch batteries or 9 volt batteries for non-implantable embodiments. Other battery types known in the art of implantable medical devices may also be used. Where MD 200 is external to the patient's body, for example, power supply 280 may comprise a photo-voltaic or solar cell.

The MD 200 may also comprise a communication unit 260 capable of facilitating communications between the MD 200 and various devices. In particular, the communication unit 260 is capable of providing transmission and reception of electronic signals to and from an external unit 270, such as computer 150 and a wand 155 that can communicate with the MD 200 remotely (FIG. 1). The communication unit 260 may include hardware, software, firmware, or any combination thereof

It should be noted that any of the connections 201, 211, 277, 297, or that between external unit 270 and database unit 250 may be wired or wireless, as a matter of routine skill for the person of ordinary skill in the art having the benefit of the present disclosure.

Also, the MD 200 may comprise a cost of testing unit 261 capable of determining at least some of the costs associated with responsiveness testing of the patient. For example, the cost of testing unit 261 may calculate power consumption by the various units associated with testing that are described herein, consumption of computational resources by the various units associated with testing, or the like. This information may be useful to the clinician in order to allow him or her to adjust the number, difficulty, or other parameters of the responsiveness tests administered to the patient.

The MD 200 may also comprise one or more sensor(s) 212 coupled via sensor coupling 211 (which may comprise a lead or an inductive coupling) to the MD 200. The sensor(s) 212 are capable of receiving signals related to a body parameter, such as an autonomic or neurologic index, and delivering the signals to the MD 200. In a particular embodiment, the sensor(s) 212 deliver the signals to the controller 210, where they may be processed by the processor 215 and/or stored in the memory 217, and/or routed to the triggering event indication receiving unit 265, as discussed below.

Exemplary sensor(s) 212 include electrocardiography (EKG) devices, accelerometers, inclinometers, pupillometers, face or body temperature monitors, skin resistance monitors, and/or sound and pressure sensors, among others.

In one embodiment, the sensor(s) 212 may be the same as stimulating electrode(s) 226, 228. In other embodiments, the sensor(s) 212 are separate structures that may be placed in, on, or near a particular organ, tissue, nerve, or blood vessel of the patient, or outside the patient's body, such as on the patient's skin or in the patient's environment.

In one embodiment, the MD 200 may comprise a triggering event indication receiving unit 265 that is capable of receiving signals related to a triggering event. The triggering event indication receiving unit 265 may be capable of performing any necessary or suitable amplifying, filtering, and performing analog-to-digital (A/D) conversions on the received signals to determine whether a triggering event has occurred. The triggering event indication receiving unit 265, in one embodiment, may comprise software module(s) that are capable of performing various interface functions, filtering functions, etc., to determine whether a triggering event has occurred. In another embodiment the triggering event indication receiving unit 265 may comprise hardware circuitry that is capable of performing these functions. In yet another embodiment, the triggering event indication receiving unit 265 may comprise hardware, firmware, software and/or any combination thereof.

The triggering event indication receiving unit 265 may determine whether a triggering event occurred, wherein the triggering event is selected from the group consisting of a) an indication from a medical event detection algorithm that a medical event is occurring or is imminent; b) a manual signal to administer the responsiveness test to the patient; or c) a command to administer a responsiveness test to the patient in the absence of an indication from a medical event detection algorithm that a medical event is occurring or imminent.

The triggering event for testing responsiveness may include one or more of a) an indication from a medical event detection algorithm, based upon one or more body parameters of the patient, that a medical event relevant to the patient's condition is occurring or is imminent (e.g., detection of a future, imminent, or on-going epileptic seizure or other medical event using heart or other autonomic indices or brain activity of the patient, which may also be described as a positive or affirmative output of a medical event detection algorithm (POMEDA)), b) a manual signal from a patient, caregiver or physician to administer the responsiveness test to the patient, or c) a command to administer a responsiveness test to the patient in the absence of an indication from a medical event detection algorithm that a medical event is occurring or imminent (e.g., a command to administer a responsiveness test to the patient during a negative output of a medical event detection algorithm (NOMEDA)). The command provided in the absence of a medical event detection event may be based upon circadian or ultradian rhythms of the patient, past medical event history of the patient (e.g. times of day, week, or month when medical event probability exceeded a specified value), expiration of a random or pseudorandom timer or similar events. Elapse of a periodic, random, or pseudorandom time period may be used to present a responsiveness test to the patient in response to a NOMEDA so as to: a) establish a representative baseline (e.g., non-seizure) performance for comparison (statistical) with that associated with medical events, including a post-medical events period during which performance or responsiveness may remain impaired for some time; b) avoid anxiety or conditioning of the patient to expect a medical events whenever a test is administered, and c) to minimize biasing test responses.

The end of a time period may be determined either by comparing the current absolute time with a time previously determined to indicate the end of the time period, or by incrementing a counter of time units until a value previously determined to indicate the end of the time period is reached. For example, the time period may be programmed to have a predefined or random duration with a specified range, e.g., from 15 minutes to 24 hours, or may be programmed as a specific number of random timepoints within a 24 hour period or weekly period. Tests in response to a NOMEDA can be administered while the patient is awake to establish a representative baseline performance, since response times and other measures of cognitive performance vary as a function of circadian rhythms. For statistical purposes, it may also be desirable to administer tests at about the same time of day on consecutive days; and/or at about the same time of a day as a medical event that occurred on a previous day; among other possibilities. To ensure statistical validity, in one embodiment the logic associated with administering the test in response to what is believed to be a NOMEDA may verify that the output of any medical event detection algorithm is, in fact, negative (i.e., that no medical event is occurring or imminent, as contrasted with the simple expiration of a timer) before the test is applied and logged as having occurred in response to a NOMEDA.

The previously determined time (for testing purposes) may be set at any point prior to the end of the time period, and may be programmed or reprogrammed by the manufacturer or the practitioner. For example, the ratio of “non-algorithm-triggered” (i.e., manual or NOMEDA) to “algorithm-triggered” (i.e., POMEDA) events can be programmed, e.g., about equal POMEDA and NOMEDA testing, 50% more (or less) NOMEDA testing than POMEDA, or increased testing (both POMEDA and NOMEDA triggered) as a function of when (e.g., time of day, time of week, etc.) the patient historically has an increased probability of medically relevant events.

For POMEDA-triggered testing, a change in an autonomic or neurologic index may be determined by receiving a value related to an autonomic or neurologic index and comparing it with a previously determined value. The autonomic or neurologic index value may be determined by analyzing at least one set of signals received from the patient and selected from the group consisting of cardiovascular signals, breathing signals, pupillary signals, skin signals, blood pressure, among others. Additionally or alternatively, neurological signals, such as those generated by the brain or cranial nerves or body kinetic signals (e.g., signals generated by motion of the patient's body as determined by an accelerometer or inclinometer) may be used for detecting medically relevant events, such as epileptic seizures.

For example, the autonomic or neurologic index value(s) used to determine whether a medical event has occurred may be the heart rate, a change in the heart rate, or the rate of change in heart rate, and the triggering event may be a heart rate above a first previously determined value, a heart rate below a second previously determined value, or a rate of change of heart rate above a third previously determined value, a heart rate variability above or below a fourth or fifth previously determined value, among others. Other cardiovascular indices values include, but are not limited to, blood pressure, heart sounds, heart rhythm, heartbeat wave morphology, heartbeat complex morphology, or the shape of the deflection of the thoracic wall as the heart apex beats against it, among others. Such cardiovascular index values can be detected by electrocardiography, blood pressure monitors, a microphone, or apexcardiography, among others.

For another example, the autonomic or neurologic index value(s) used to determine whether a medical event has occurred may be related to the respiration (breath) rate, and the triggering event may be a respiration rate above a first previously determined value, a respiration rate below a second previously determined value, or a rate of change of respiration rate above a third previously determined value, among others. Other respiratory index values include, but are not limited to, respiration pattern, airflow velocity, respiration amplitude (tidal volume), oxygen saturation, arterial gas concentrations, and blood pH, among others. Such respiratory index values can be detected by techniques and apparatus known to the person of ordinary skill in the art.

For still another example, the autonomic or neurologic index value(s) used to determine whether a medical event has occurred may be related to one or more skin signals, such as a change in the skin resistivity of the patient.

For another example, the autonomic or neurologic index value(s) used to determine whether a medical event has occurred may be related to one or more temperature signals, such as a change in the skin temperature of a part of the patient's face (e.g., face) (see Sunderam & Osorio) or a change in the core temperature of the patient.

For still another example, brain signals, such as those determinable by an EEG or ECoG may be used to determine whether a medical event has occurred, and the triggering criterion may be a value of one or more of the brain signals above a first previously determined value or below a second previously determined value.

For another example, the detection criterion may be related to one or more body kinetic signals. The body kinetic signal may be determinable by electromyography, an accelerometer, and/or an inclinometer, and the triggering criterion may be a value of the body kinetic signal indicative of the body's (or of a portion thereof such as an arm or a leg) acceleration, direction, position, amplitude or force of movements.

For yet another example, the index value(s) used to determine whether a medical event has occurred may be related to one or more cranial nerve signals.

For yet another example, the index value(s) used to determine whether a medical event has occurred may be related to one or more autonomic nerve or ganglia signals.

For still a further example, a plurality of autonomic and/or neurologic (e.g., brain, cranial nerve, or kinetic) index value(s) may be used to determine whether a medical event has occurred. For example, the triggering event may be a finding that the patient's heart rate is above a threshold value at a time when a body kinetic signal shows the patient's body orientation is reclined or when it indicates the patient stopped moving.

For POMEDA-triggered testing, the algorithm used may be one that determines a probability of a medical event and yields a positive output if the probability of the medical event exceeds a threshold. For example, one or more autonomic or neurologic indices may be assigned a weight, such as in the range 0 to 1, based on its history of sensitivity and/or specificity regarding the patient's medical condition; the time of day, time of week, time or month, time of year, the patient's wake/sleep status, the patient's physical activity level, the patient's current or recent food intake; or the like. The index value(s) may be analyzed to produce an output value, e.g., a probability, p. If the probability exceeds a threshold, a positive output may be yielded by the algorithm.

Whether a seizure occurred may be determined by analyzing a change in an autonomic or neurologic index value, temporal or other patterns, or morphologies, such as those discussed above; by receiving an input from a medical event detection algorithm; or by receiving an input from a clinician or knowledgeable layperson who observes an electroencephalographic or clinical onset of a seizure. Such analysis is known, for example, from work by the present inventors, such as U.S. Pat. Nos. 7,457,665; 6,961,618; and 6,549,804, hereby incorporated by reference herein.

The triggering event indication receiving unit 265 is capable of receiving an indication of a triggering event and communicating such receipt to the controller 210. Based upon the indication received by the triggering event indication receiving unit 265, a responsiveness testing unit 285 may administer a test of responsiveness to the patient.

“Responsiveness” is used herein to refer to any response made by a patient upon exposure to a stimulus.

“Motor function” is used herein to refer to a function actuated by the contraction of at least one muscle of the patient.

In one embodiment, responsiveness is part of a cognitive function. “Cognitive function” is used herein to refer to an action that indicates to an observing adult of at least average intelligence and mental health that the patient is purposefully implementing a behavior in pursuit of an objective. Examples of cognitive functions include, but are not limited to, attention, short-term memory, long-term memory, language fluency, visuospatial awareness, abstract reasoning, or two or more thereof.

In another embodiment, the test of responsiveness can test the patient's reflex function.

In a system as complex as the human body, the person of ordinary skill in the art would understand that for a cognitive function to be observed and/or measured, the cognitive function implements a motor function.

In certain embodiments, a positive test of motor function may reveal purposive behavior was performed by the patient to yield that positive test, i.e., a positive test of motor function in these embodiments may be further taken as a positive test of cognitive function. The converse is not necessarily true. In other words, a negative test of cognitive function may be coincident with various motor functions, such as non-purposive movement of limbs, non-purposive vocalization, etc. For example, a patient with cognitive impairment may experience certain reflex motions or “automatisms” that are not to be confused as positive evidence of cognitive function.

In certain embodiments, a test of responsiveness may distinguish between alertness (ability to orient to new stimuli) and attentiveness (ability to engage and decode stimuli).

A “test of responsiveness” is any combination of one or more outputs to the patient (such as one or more outputs provided by output device 198) and one or more responsiveness inputs received from the patient (such as one or more inputs provided by input device 199 or patient input unit 275). Typically, it will take some length of time for the human brain to decode a stimulus and encode a response, whether correct or incorrect.

In the embodiment shown in FIG. 2A, the function of the input device 199 (FIG. 1B) is performed by patient input unit 275. The patient input unit 275 may include a magnetic signal input sensor (such as, for example, a Reed switch) or a tap input sensor, among others. In one embodiment, the patient input unit 275 may also be used to allow the patient to request of the medical device 200 alterations in a therapy regimen, such as to relieve an acute symptom of the patient's disease, to intervene with the intent of forestalling an medical event, or to minimize adverse effects of the therapy at particular times.

Results of the test of responsiveness include, but are not limited to, the correctness of an input and the time required by the patient to provide the input after receiving the output. From the various results of the test, one or more measures of the patient's responsiveness may be calculated, as will be discussed in more detail below.

In certain embodiments, one or more autonomic and/or neurologic indices may give information relating to the patient's attention and/or effort given to the test. For example, pupillary information, such as blink frequency, blink duration, fixation frequency, dwell time, saccadic extent, and mean pupil diameter, any or all of which may be normalized for ambient lighting or other environmental factors, and/or to a baseline, can be used by the person of ordinary skill in the art to determine whether the patient is paying attention to the test. For another example, electromyography (EMG) may give information about the patient's facial muscle tone, which can be used by the person of ordinary skill in the art to gauge the patient's effort given to the test.

In one embodiment, the test comprises the serial and simultaneous presentation of a pair of visual “stimuli” (such as the letter “A” and a square “□”) on a output device 198 (FIG. 1B). The position of the letter on either half of the screen (i.e., left “A □”, or right “□A”) may be randomly chosen for each presentation and the patient instructed to immediately make an input according to which side of the screen (left or right) on which the letter A appears. In one embodiment, the input comprises pressing one of two buttons, one button representing left and the other representing right. A correct press may then correspond to pressing the button ipsilateral to the side of the screen where the letter A was displayed.

Test complexity may be increased (as needed) by displaying the stimuli in the vertical plane and alternating this (randomly) with the horizontal display or by randomly switching between stimuli during a test. For example, the patient may be instructuted to press the button ipsilateral to the letter, except when the square is filled with a solid color (other than the background color), in which case the button ipsilateral to the side where the filled square appears should be pressed. This test may be triggered by a POMEDA or in response to a NOMEDA to allow comparison and quantification of changes in performance during algorithm-triggered and non-algorithm-triggered periods.

During the test, as soon as the subject presses either button, or after a maximal presentation time (e.g., 1 s) had elapsed, in case of no response, each stimulus presentation is removed (resulting in a blank screen) until the next stimuli. At the end of each presentation, a random timer (with appropriate upper and lower limits) can be set, the expiration of which triggers the next presentation. A fixed number of stimuli, such as 36, may be presented in each testing session, or the number of stimuli may be made a function of event duration and/or severity as derived from an autonomic or neurologic index value, or the number of stimuli may be made a function of results of one or more previous stimuli (e.g., if the patient's results show substantially full cognitive function for one or more trials of an seizure test, the number of further stimuli may be increases to better assess cognitive performance). Inter-trial presentation time intervals may, in one embodiment, be randomly chosen from a finite set of time intervals, such as from the set {0.5 s, 1.0 s, 1.5 s, 2.0 s}, to minimize adaptation and better assess performance.

Although a single test may be administered to the patient, such as during a medical event, such as a seizure, in other embodiments, multiple identical or different tests may be administered. In one embodiment, when the patient's medical condition is epilepsy, tests can be administered at a plurality of times, wherein at least one of the plurality of times is ictal (i.e., during an epileptic medical event) and at least one of the plurality of times is nonictal (i.e., at a time not during an epileptic medical event). A test administered at a nonictal time may be referred to herein as a “baseline” test. In embodiments wherein the patient's medical condition is not epilepsy, tests may similarly be administered during a medical event, not during a medical event, and/or during other times.

The baseline test or the difficulty thereof may be adjusted by the clinician in view of the patient's general condition, e.g., a pediatric patient, a geriatric patient, or a mentally challenged patient may require a simpler baseline test, if the patient has difficulty completing a more complicated baseline test in a short period of time.

The test may also be administered to patients suffering from neurological or psychiatric disorders and/or to subjects free from such disorders.

The patient's responsiveness may be determined by responsiveness determination unit 287. The responsiveness may be determined from one or more values extractable from results of the test, such as the correctness of an input (such as a fraction or percentage of correct inputs), or the time required by the patient to provide the input after receiving the output, among others.

In one embodiment, multiple separate tests may be administered having various difficulty levels or the level of difficulty of a single test may be increased gradually or suddenly or different levels of difficulty may alternate randomly. For example, in one embodiment, a first test of responsiveness having a first difficulty level is selected and administered, and, based on the results of the first test, a second test of responsiveness having a second difficulty level is selected and administered. For comparison purposes, these tests could be administered to patients during POMEDA periods and NOMEDA periods (e.g., for epilepsy patients, during seizure and non-seizure conditions), and comparisons could be performed between tests of equivalent difficulty.

In one embodiment, the difficulty and duration of the degree of responsiveness test may be optimized according to the cost of detection or the clinician's desired balance of sensitivity (low false negative rate) and specificity (low false positive rate), and/or to account for changes in event severity over time. Doing so may require co-analysis of the degree of responsiveness test results with another indicator of a medical event, such as can be read from an autonomic (e.g., heart rate) or neurologic, (e.g., EEG) of the patient, or some other indicator that may exist or be developed.

In one embodiment specific to epilepsy patients, at the termination of a degree of responsiveness test, whether triggered by a POMEDA or not, the patient may be asked if he or she just had a seizure. This is a way to classify seizures as clinical or subclinical and an indirect way to validate detections; the patient's time-stamped response will be routed to the local database unit 255 and/or the memory 217, where it will be cross-referenced with an autonomic or neurologic index value if one indicative of a seizure exists at about the time the patient's response was recorded. If the patient indicates a seizure has occurred and an index indicative of a seizure exists about the time of the response (i.e., the test was triggered by a POMEDA) it is classified as clinical. If the patient responds in the negative and the index value is indicative of a seizure, it is classified as subclinical. If the patient responds that a seizure has occurred and none of the indices supports it (i.e., the test was triggered by a NOMEDA), a false negative detection is recorded. This provides quantitative information about the status of the patient's condition and about therapeutic and/or diagnostic efficacy that is not currently available and complements that provided by seizure frequency measures. It may also allow qualitative validation of other seizure severity measures. Additionally, interrogating the patient after a test has been administered, may help blind the patient to whether the test was triggered by a POMEDA, and thus whether a seizure is occurring or imminent. Asking the patient if a seizure occurred after administration of a test may be programmed to take place after each test until a sample sufficiently large to support statistical analyses has been collected, and updated based on the status of the patient's condition and the response to therapy and/or diagnosis.

In one embodiment, the responsiveness determination unit 287 can perform a quantification of one or more measures relating to the test results and/or the patient's responsiveness. For example, if the test of responsiveness encompasses tracking the response time of the patient to deliver a correct answer to a stimulus (viz., speed of response), the response time of a plurality of trials can be tracked and quantified, such as by use of measures of central tendency. The percent of correct responses may be also quantified and used alone or in conjunction with the speed of response to mark the time of onset of lack of responsiveness, and its duration.

More specifically, based on the patient's responsiveness, the responsiveness parameter unit 288 may determine a time of occurrence of a change in the patient's responsiveness, a duration of a change in the patient's responsiveness; a magnitude of a change in the patient's responsiveness, a time interval from the indication of triggering event occurrence to a change in the patient's responsiveness; a type (e.g., motor, cognitive, or both) of change in the patient's responsiveness, a determination of medical event severity for the patient; a formulation of a prognosis for the patient; an estimation of a risk of injury or death for the patient; and an assessment of efficacy of a therapy for the patient's medical condition, or some other parameter. The determination may require acquisition of an adequate sample or samples of patient results of responsiveness tests. For example, the responsiveness parameter unit 288 may compare the patient's responsiveness at a first time to a database of time-ordered prior measures of responsiveness (which may be patient-specific or data from a plurality of patients), which may be stored in local database unit 255 or database unit 250, and then review the set of measures in the database taken from time points after each appearance in the database of a measure reflective of the patient's responsiveness at the first time to determine whether the patient is having a medical event, the patient's baseline responsiveness, the patient's long-term prognosis, or the like.

Strictly speaking, in embodiments where the medical condition is epilepsy, the seizure severity calculated here relates to loss of function, not a seizure as an electroencephalographic event. However, the duration, severity, or both of a loss of function may be used as reasonable approximations and/or indicators of the duration and severity of at least some types of seizure.

For example, if a plurality of trials of a responsiveness test are triggered by a POMEDA, the medical event duration may be approximated as the time between the first iteration of the test in which the patient responded incorrectly or failed to respond to three consecutively presented stimuli and the next subsequent iteration of the test in which the patient responded correctly. Another relevant measure of severity—the latency from seizure detection to loss of function—may be determined by the time between an indication of event occurrence (e.g., a POMEDA) to the first iteration of the test in which the patient responded incorrectly. Changes in the latency from seizure detection to loss of function over time may be used to determine whether the patient's disease state is improving, worsening, or remains about the same.

For example, if the speed of response, the correctness of the response, and/or the difficulty of response are logged for each of a plurality of iterations of the test, the sum of response times for iterations above the baseline (optionally weighted by the correctness and/or difficulty) and the times at which those iterations were administered can be used to calculate an area under the curve, from which a medical event severity can be, in full or in part, approximated.

In various embodiments, one or more of the units or modules described above may be located in an external unit 270 or a remote device 292, with communications between that unit or module and communication unit 260 in the MD 200 taking place via a link 277, which may comprise a lead, an inductive RF or similar wireless coupling, a Bluetooth or other wireless data transfer coupling, etc. For example, in one embodiment, as shown in FIG. 2B, a triggering event indication receiving unit 265, a responsiveness testing unit 285, a responsiveness determination unit 287, and a responsiveness parameter unit 288 may be located in an external unit. In embodiments in which no therapy unit 220 or stimulation electrodes 226, 228 are provided, all of the functional modules may be provided in an external unit.

In one embodiment, the external unit 270 may comprise a local database unit 255. Optionally or alternatively, the external unit 270 may also be coupled to a database unit 250, which may be separate from external unit 270 (e.g., a centralized database wirelessly linked to external unit 270). The database unit 250 and/or the local database unit 255 are capable of storing various patient data. In certain embodiments, the memory 217 is also capable of storing various patient data. These data may comprise time stamped: a) patient responsiveness data, including test results and measures of responsiveness, b) number of detections (i.e., POMEDAs) and their severity expressed numerically as the product of peak index value and the time (in sec or min) which the index value spends above a baseline; c) medical event classification (e.g., whether a seizure is clinical or subclinical, and if clinical, if it is simple partial, complex partial, or generalized); d) therapy parameter data; e) disease status as assessed with autonomic and neurological indices; f) injury risk; h) event button presses or patient input, all data being updated as its flow rate demands (as will be discussed in more detail below). The time stamp may have any particular desired granularity, such as to the hundredth of a second. The database unit 250 and/or the local database unit 255 may comprise data for a plurality of patients, and may be organized and stored in a variety of manners, such as in date format, severity of disease format, latency (time difference) between a detection event (i.e., a POMEDA) and a loss of responsiveness as indicated by the failure of the patient to respond to one or more test stimuli, etc. The database unit 250 and/or the local database unit 255 may be relational databases in one embodiment. The database unit 250 and/or the local database unit 255 may store various patient data.

In one embodiment, the database unit 250 and/or the local database unit 255 allow the patient, a caregiver, a medical practitioner, or another interested person to follow the patient's responsiveness under various changing conditions, such as under various disease conditions (e.g., for epilepsy patients, during preictal, ictal, and/or postictal times), various times of day, month, and/or year, various therapy parameters, etc.

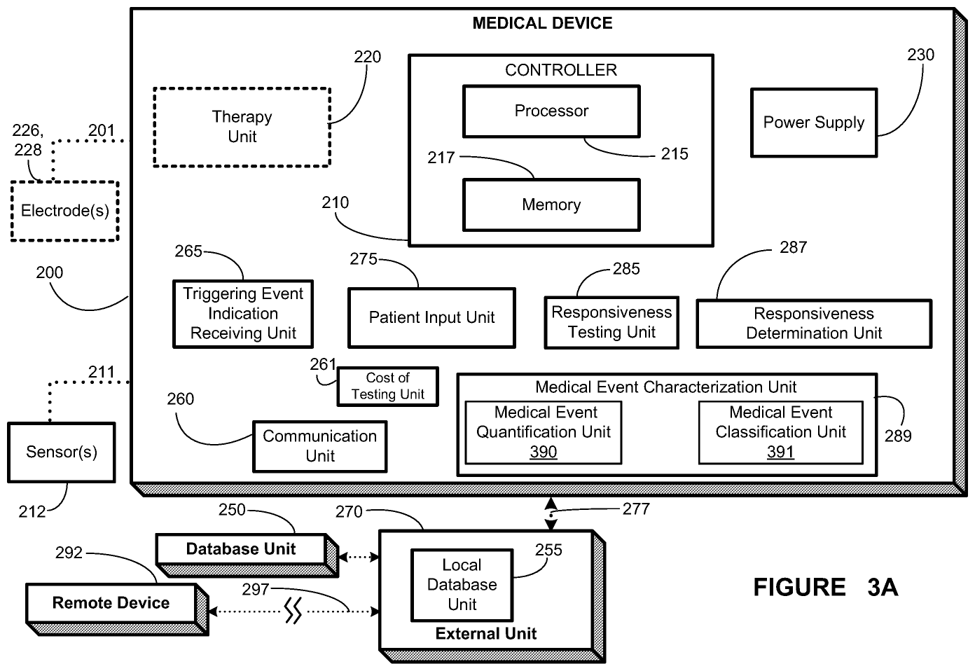

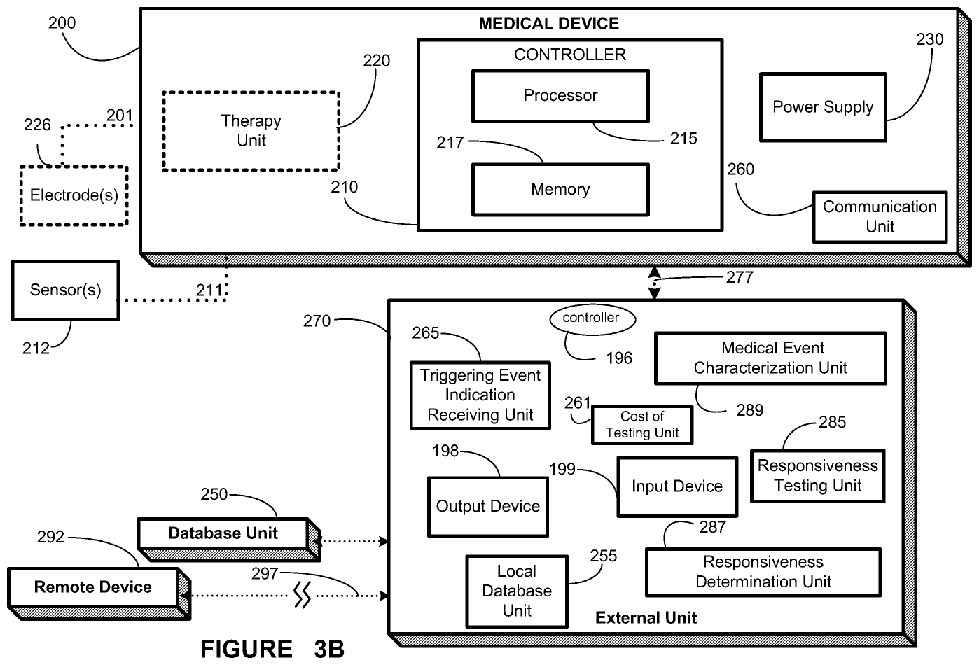

FIGS. 3A-3B contain many like elements to FIGS. 2A-2B, and those like elements will not be described further. FIGS. 3A-3B contain a medical event characterization unit 289, comprising a medical event quantification unit 390 and a medical event classification unit 391. Based on either or both of the patient's responsiveness and one or more other indicators of a medical event (e.g., a POMEDA from an algorithm based on the patient's heart rate or heart rate variability, among others), the medical event quantification unit 390 may determine a duration or a severity of a medical event and/or the medical event classification unit 391 may classify the medical event by duration, severity, and/or estimated type (e.g., for seizures, partial or generalized, simple or complex, etc.). For example, the medical event characterization unit 289 may determine a series of measures in a database of time-ordered values, which may be stored in local database unit 255 or database unit 250, in which many or all of the measures reflected a medical event, and determine the duration of a medical event by comparing time-stamps of the first and last values in the series comparable to a measure of the patient's baseline responses. A severity of a medical event may, for example, be calculated from the sum, average, median, mean, nth percentile, or area under the curve of one or more measured values in the series.

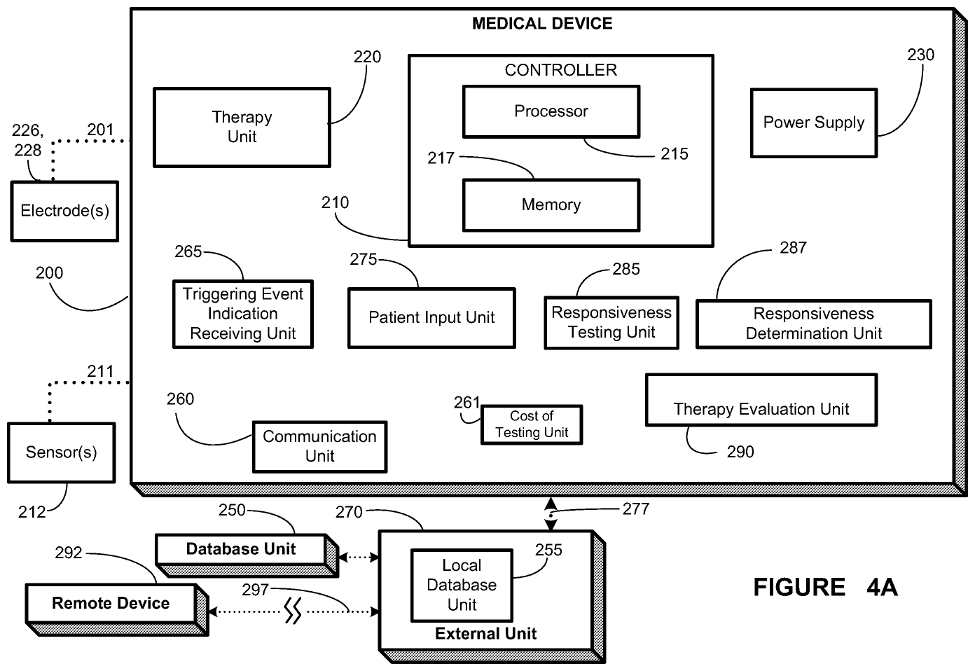

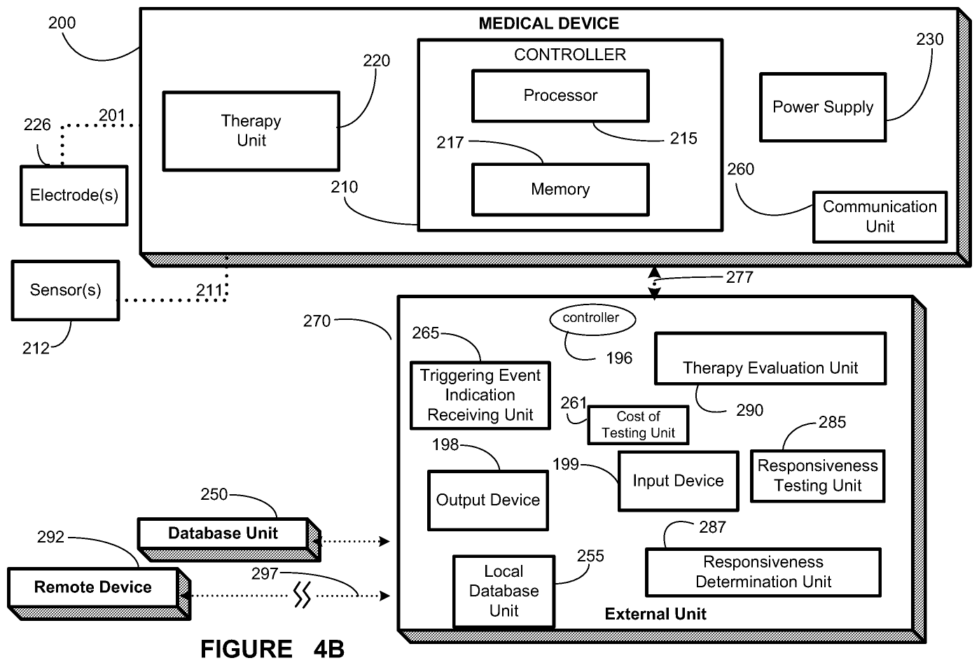

FIGS. 4A-4B contain many like elements to FIGS. 2A-2B or FIGS. 3A-3B, and those like elements will not be described further. FIGS. 4A-4B illustrate an embodiment of the invention having a therapy evaluation unit 290. Therapeutic efficacy of a therapy provided by a therapy unit 220 can be assessed by determining if there is a decrease in the severity or duration of the patient's loss of responsiveness over time. Decreases in value of any of the autonomic or neurologic indices--including electrical activity recorded directly from the brain--do not provide direct or reliable information about the state of a patient's responsiveness. Based on the patient's responsiveness, the therapy evaluation unit 290 may determine an efficacy of a therapy for a patient's medical condition. For example, the therapy evaluation unit 290 may compare a measure of the patient's responsiveness when a therapy is administered by a therapy unit 220 compared to when therapy was not administered. Alternatively, the therapy may be administered by other than a therapy unit 220, e.g., by the patient's oral ingestion of a medication, or the like. These comparisons are performed by the therapy evaluation unit 290 using measures in a database of time-ordered values, which may be stored in local database unit 255 or database unit 250. For example, the measure may be a correct response time to a test, optionally weighted by difficulty of the test, and optionally smoothed as the mean, median, or the like of a series of values. If the correct response time(s) for administrations of the test is/are lower when a therapy is administered than when it is not, the therapy's efficacy may be quantified, for example, as the percent reduction of the duration and/or severity of the patient's loss of responsiveness. Efficacy may also be quantified as the change in the latency time from medical event detection (as determined by, e.g., a POMEDA) to loss of function (e.g., the first iteration of the test in which the patient responded incorrectly) over the course of a desired time interval such as weeks, months, or years.

A therapy evaluation unit 290 may be desirable for inclusion in a medical device system wherein the medical device system delivers a therapy for a seizure event to the patient. In one embodiment, the therapy for the seizure event is selected from the group consisting of electrical stimulation of a cranial nerve of the patient, thermal manipulation of the cranial nerve of the patient, electrical stimulation of the brain of the patient, thermal manipulation of the brain of the patient, delivery of a chemical agent to the patient via the bloodstream, the cerebrospinal fluid or directly to brain tissue, performance of a motor task, performance of a perceptual task, performance of a cognitive task, and two or more thereof.

In one embodiment, the therapy evaluation unit 290 may be incorporated into the medical device 200 (see, for example, FIG. 4A).

Though not shown, the person of ordinary skill in the art will understand a medical device system according to the present invention may comprise any two or all three of a responsiveness parameter unit 288, a medical event characterization unit 289, and a therapy evaluation unit 290. For example, the medical device system may comprise both the medical event characterization unit 289 and the therapy evaluation unit 290, and the therapy evaluation unit 290 may incorporate in its therapy evaluation event duration and/or event severity values reported by the medical event characterization unit 289.

One or more of the blocks illustrated in the block diagram of the MD 200 in FIGS. 2-4 may comprise hardware units, software units, firmware units, or any combination thereof.

Additionally, one or more blocks illustrated in FIGS. 2-4 may be combined with other blocks, which may represent circuit hardware units, software algorithms, etc. Additionally, any number of the circuitry or software units associated with the various blocks illustrated in FIGS. 2-4 may be combined into a programmable device, such as a field programmable gate array, an ASIC device, etc.

The medical device system of one embodiment of the present invention provides for software module(s) that are capable of acquiring, storing, and processing various forms of data, such as patient data/parameters (e.g., demographic data, physiological data such as autonomic and neurologic index values, such as heart rate or EKG morphology changes or breathing rate or pattern changes, among others, disease status (progression, regression, or stabilization), quality of life data, etc.). In one embodiment, the software modules(s) are further capable of acquiring, storing, and processing therapy parameter data. Therapy parameters may include, but are not limited to, electrical signal parameters that define therapeutic electrical signals delivered by the device, medication parameters, and/or any other therapeutic treatment parameter. In an alternative embodiment, the term “therapy parameters” may refer to electrical signal parameters defining the therapeutic electrical signals delivered by the IMD. Therapy parameters for a therapeutic electrical signal may also include, but are not limited to, a current amplitude, a pulse width, a pulse shape, a degree of charge balancing, a frequency, a pulse train pattern, an on-time, an off-time, etc.

In one embodiment, the present invention may include coupling of at least one electrode to each of two or more cranial nerves. (In this context, two or more cranial nerves mean two or more nerves having different names or numerical designations, and do not refer to the left and right versions of a particular nerve). In one embodiment, at least one electrode may be coupled to either or both vagus nerves or a branch of either or both vagus nerves. The term “operatively” coupled may include direct or indirect coupling. Each of the nerves in this embodiment or others involving two or more cranial nerves may be stimulated according to particular activation modalities that may be independent between the two nerves.

In one embodiment, the communication unit 260 is capable, based on the patient's responsiveness, of instructing an external device to change an operating state thereof. For example, the external device may be an automobile or other vehicle which the patient is driving, and the change in the operating state thereof may be stopping, putting the transmission into neutral, applying a parking brake, etc., or two or more thereof. For another example, the device may be a power tool, such as a circular saw, table saw, jigsaw, chain saw, power sander, lawn mower, weed trimmer, tiller, cultivator, etc., and the change in the operating state thereof may be stopping its motor or disengaging its cutting or grinding parts from the motor or a drive coupled to the motor. For another example, the device may be an oven, stove, toaster, microwave oven, or other kitchen applicants. For an additional example, the device may be a faucet, a drain, or a gate restricting access to a stairwell, balcony, swimming pool, or other location where a non-responsive person would be at risk of bodily harm or death.

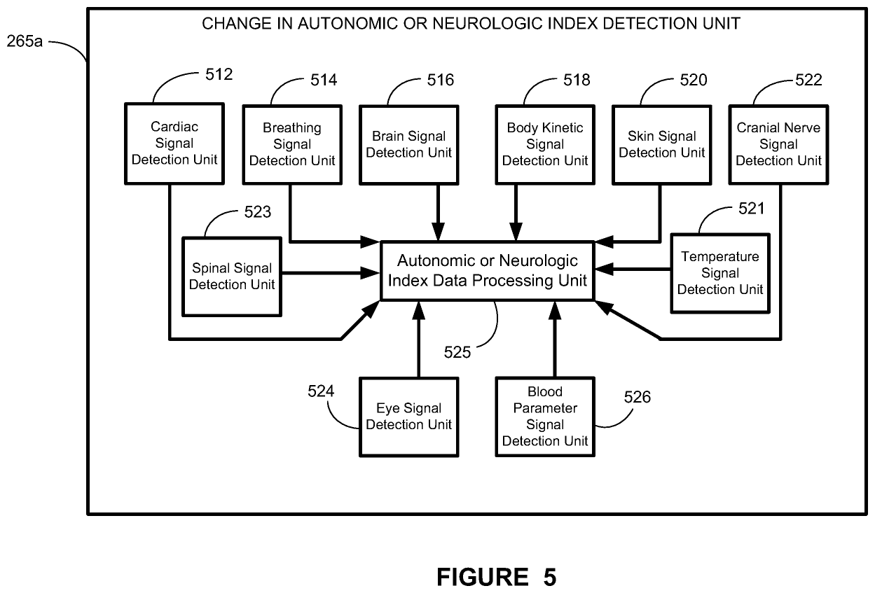

Turning now to FIG. 5, a change in autonomic or neurologic index detection unit 265a is shown. In certain embodiments, autonomic or neurologic index detection unit 265a can trigger an administration of the test by providing an appropriate instruction to triggering event indication receiving unit 265 (FIGS. 2A-4B). The change in autonomic or neurologic index detection unit 265a may comprise one or more signal detection units, such as a cardiovascular signal detection unit 512, a breathing signal detection unit 514, a brain signal detection unit 516, a motor or kinetic signal detection unit 518, a skin signal detection unit 520, a temperature signal detection unit 521, a spinal signal detection unit 523, a cranial or peripheral nerve signal detection unit 522, an eye (pupil or eyelid) signal detection unit 524, and/or a blood parameter signal detection unit 526. The cardiovascular signal detection unit 512 is capable of detecting one or more of various cardiovascular-related signals, including but not limited to electrocardiogram (ECG) signals, heart rate (HR) signals, and heart rate variability (HRV) signals. The breathing signal detection unit 514 is capable of detecting one or more of various breathing/respiratory signals of a patient, including, but not limited to, breath rate (BR) signals, air flow signals, and tidal volume signals. The breathing/respiratory parameters sensed by the unit 514 may include, but is not limited to, air flow measurements, volume measurements, transthoracic inductance, impedance plethysmogrphs, thoracic circumference, and pneumatic respiration, among others. Some embodiments of the unit 514 may include at least one of a spirometer, a nasal thermocoupler measurement device, a strain gauge, a pneumatic respiration transducer, an impedance measurement device, and/or other devices capable of detecting respiratory signals.

The brain signal detection unit 516 is capable of detecting one or more of various brain signals, including, but not limited to, EEG signals, field potentials or multiunit activity; fast neuronal oscillations (>100 Hz); near DC or DC potentials, event-related potentials, neurotranmitter concentrations, ionic concentrations, pH, glucose concentrations, free radicals and/or other brain signals known to those skilled in the art. The body kinetic signal detection unit 518 may include an accelerometer, an inclinometer, and/or other kinetic or force measurement devices capable of detecting movement in one or more areas or limbs of the patient's body. The skin signal detection unit 520 is capable of detecting one or more of various skin parameters, such as impedance or other bioelectrical measurements relating to the skin, sweat amount, sweat chemical composition, etc. The cranial nerve signal detection unit 522 is capable of detecting one or more of various signals relating to cranial nerves, such as amplitude, rate and direction of action potential traffic, type of fiber (by size and presence or absence of myelinization) activated, polarity, transmembrane voltage parameter, etc. The temperature signal detection unit 521 is capable of detecting one or more of various types of body temperature parameters, including, but not limited to core temperature changes, organ (e.g., brain) or body part (e.g., facial) temperature, etc. The unit 521 may include an infrared sensing device, a chemical-reaction based temperature sensing device, a direct temperature measurement device, etc. The spinal signal detection unit 523 is capable of sensing one or more of various spinal signals, including, but not limited to, motor neuron signals, sensory pathway signals, autonomic signals, etc.

The eye signal detection unit 524 is capable of detecting one or more of various signals relating to the eye, including autonomic functions including, without limitation, pupil width and dilation, eyelid movement and/or phenomena such as mydriasis, miosis, ptosis and/or hippus. The blood signal detection unit is capable of detecting one or more blood parameters including, without limitation, oxygen saturation, glucose concentration, and/or blood pH.

Each unit 512-526 present in the change in autonomic or neurologic index detection unit 265a is capable of communicating detected signals, or data generated from the detected signals, to an autonomic or neurologic index data processing unit 525, which is capable of determining a change in an autonomic or neurologic index, which may involve accessing prior autonomic or neurologic index information stored in, e.g., memory 217, local database unit 255, and/or database unit 250.

The autonomic or neurologic index data processing unit 525 may comprise one or more subunits capable of performing autonomic or neurologic index quantification, autonomic or neurologic index classification, or both. In particular, autonomic or neurologic index data processing unit 525 may process one or more autonomic or neurologic signals from units 512-526 according to one or more event detection algorithms to provide an appropriate instruction (e.g., a POMEDA or a NOMEDA) to triggering event indication receiving unit 265 (FIGS. 2A-4B) to trigger an administration of the test.

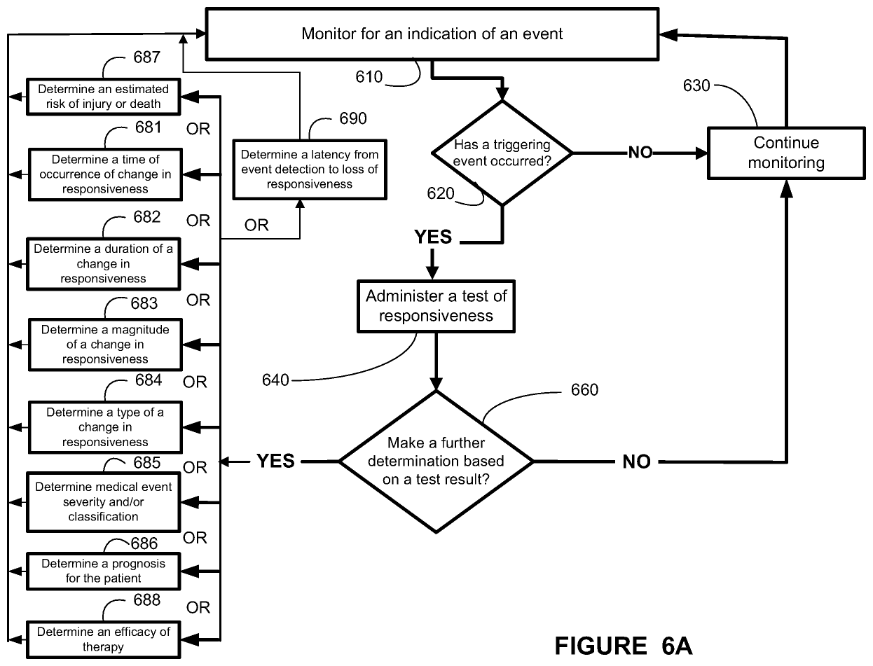

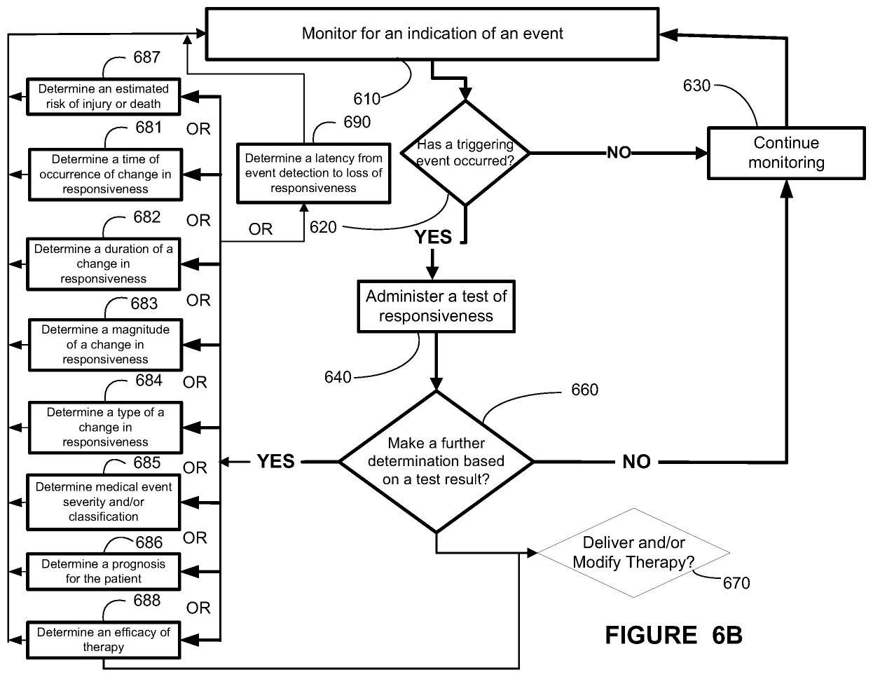

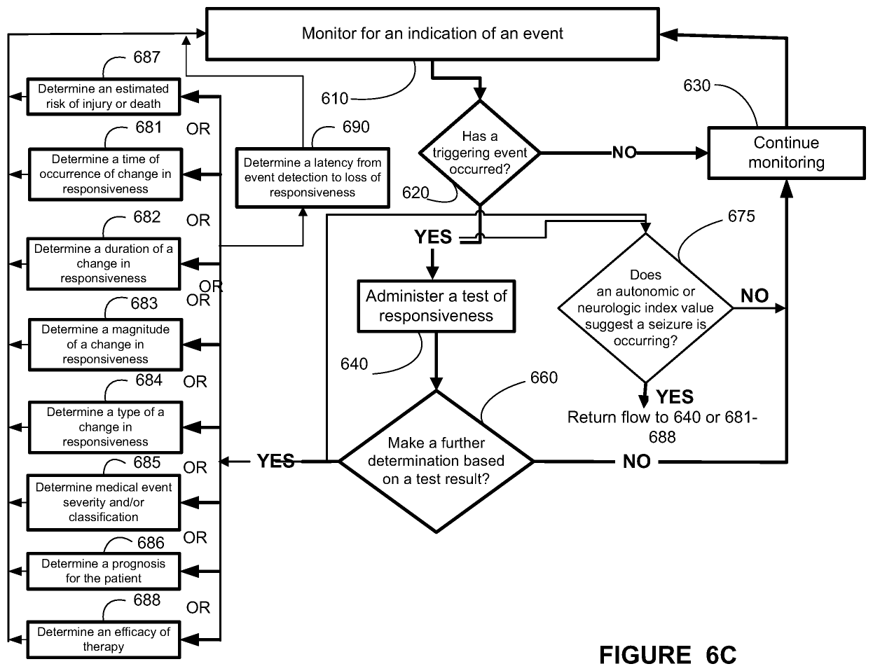

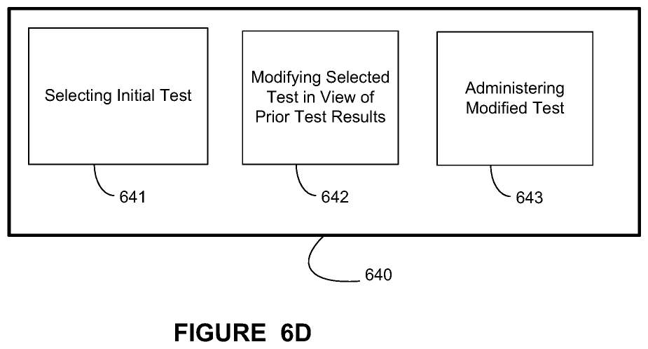

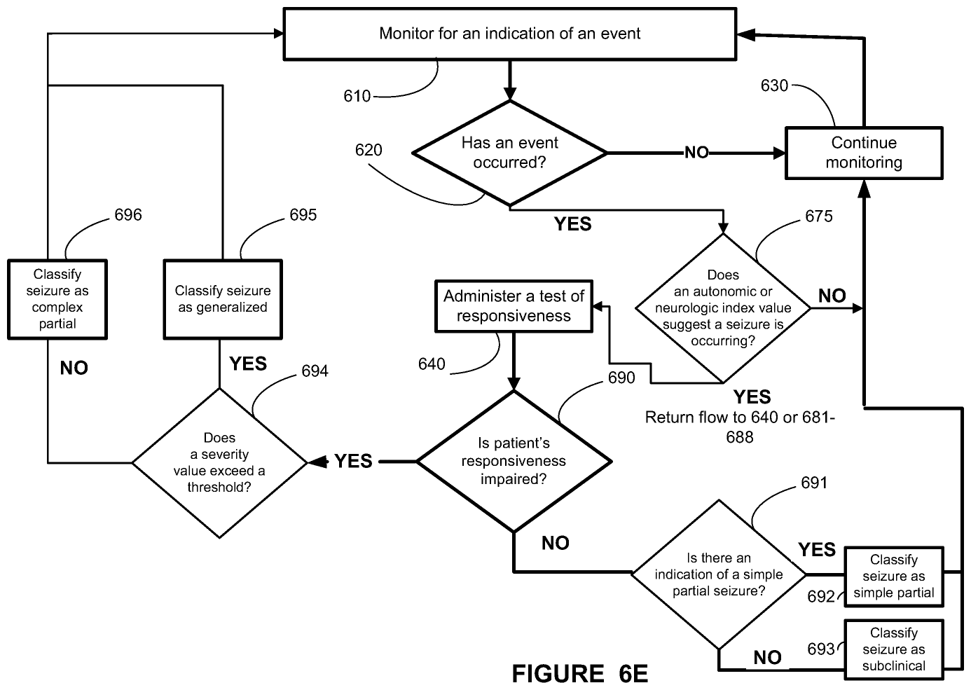

Turning now to FIG. 6A, a flowchart depiction of a method of determining a responsiveness of a patient is shown, in accordance with one illustrative embodiment of the present invention. Monitoring 610 for an indication of a triggering event, such as a signal from autonomic or neurologic index detection unit 265a, is performed. Monitoring 610 may be performed at any desirable time scale (from milliseconds to years), sampling rate (in Hz), and digital precision (in bits), which may be relatively rapid or relatively slow depending upon the nature of the signal being measured. Rapid sampling may be made for ECoG signals (e.g., about 2 KHz) or ECG signals (200 Hz to 1000 Hz), among others. Much slower sampling rates may be used for other autonomic signals, including rates as slow as once every 1 sec, 5 sec, 10 sec, 15 sec, or longer, including DC recording. Data collection or sampling may be continued or interrupted if one or more steps 640-688 are performed. Data can also be conditioned according to techniques known in the art, if desired.

After monitoring 610 is performed, a determination 620 is made as to whether a triggering event is occurring or has occurred. The triggering event may be a medical event (such as a medical event indicated by autonomic or neurologic index detection unit 265a) or a non-medical event, such as a manual signal from a patient or caregiver, or a signal from one or more random timers indicating the need for an administration of a responsiveness test. If no triggering event occurred, monitoring is continued 630. On the other hand, if a triggering event did occur, a test of responsiveness can be administered 640. In one embodiment, administration 640 of the test of responsiveness follows the description of test administration set forth above. After administration 640, a decision 660 is made as to whether a further determination should be made based on a test result. The decision parameters may be fixed in manufacture of a device or software implementing the method, reprogrammable by a practitioner during ongoing implementation of the method, and/or automatically adjustable by the device or software during ongoing implementation of the method. In one embodiment, the determination 660 may function such that a “no” decision in step 660 indicates a normal or baseline responsiveness of the patient. In this embodiment, following a “no” decision, monitoring may be continued 630.

The factors considered in the determination 660 may be adjusted to increase or decrease the specificity, sensitivity, or both of the process.

If a further determination is to be made based on the test result, i.e., upon a “yes” decision in step 660, one or more determinations 681-690 may be made. In the depicted embodiment, these determinations 681-690 (listed in no particular order) include (681) a time of occurrence of a change in the patient's responsiveness, (682) a duration of a change in the patient's responsiveness; (683) a magnitude of a change in the patient's responsiveness, (684) a type (e.g., motor or cognitive) of change in the patient's responsiveness, (685) a determination of event severity and/or classification for the patient (e.g., a classification of a seizure into clinical or subclinical and/or a classification of a clinical seizure into simple partial, complex partial, or generalized); (686) a formulation of a prognosis for the patient; (687) an estimation of a risk of injury or death for the patient; (688) an assessment of efficacy of a therapy for the patient's medical condition; and (690) a latency from medical event detection to loss of function. Steps 681-690 may be executed in parallel (simultaneously), in the order that maximizes operational efficiency and therapeutic efficacy or in any order as required by the application. If monitoring (step 610) has been discontinued while any of the other steps (681-690) are being performed, step 610 will resume immediately upon termination of steps 681-690.

The determining steps 681-690 may be performed in their entirety before returning to monitoring step 610, but they need not be. In one embodiment, the “yes” decision in step 650 instructs an appropriate device or software to initiate determining steps 681-690, gives the device or software information gathered in steps 620, 640, and/or 660 to allow the determining steps 681-690 to be executed and perform calculations using that information, and, if needed, gives the device or software permission to access a database, such as may be stored in, e.g., memory 217, local database unit 255, and/or database unit 250. Thereafter, the device or software may implement determining steps 681-690 in parallel or in any desired order with resumed monitoring 610.

Also, it should be noted that not all medical events, and not even all epileptic seizures, are necessarily associated with impaired or abnormal responsiveness but these (and those associated with impaired responsiveness) may be associated with distinctive feelings, sensations, emotions, illusions, hallucinations, thoughts, or impulses/behaviors. In one embodiment, the approaches and methods described herein allow the classification of epileptic seizures into clinical (subjective or objective phenomenology is present) or subclinical (neither subjective nor objective phenomenology is present) seizures, wherein the clinical seizures may be further classified into simple partial, complex partial, or generalized seizures. The distinction between complex and secondary generalized may be made using other seizure severity measures (e.g., peak heart rate x duration). For simple partial seizures, one or more autonomic and/or neurologic indices and/or features from each signal thereof may be used to distinguish seizures from non-ictal tachycardia.

FIG. 6B contains many elements like to those of FIG. 6A, which will not be separately discussed here. In the embodiment depicted in FIG. 6B, upon a “yes” decision 660, and/or a determination of a latency 690, in addition to the determining steps 681-690, a decision 670 is made to deliver and/or modify therapy. For example, the decision 670 may be to initiate delivery of electrical stimulation to a neural structure, such as a cranial nerve; change one or more parameters defining an electrical signal, such as the pulse width, pulse frequency, on-time/off-time ratio, or other parameters of electrical stimulation to a neural structure, such as a cranial nerve, deliver or change the dosage of a drug administered to the patient; etc.

FIG. 6C contains many elements like to those of FIG. 6A and/or FIG. 6B, which will not be separately discussed here. In the embodiment depicted in FIG. 6C, upon either or both of the “yes” decisions yielded by decision nodes 620 and 660, the process flow may be directed to step 675, wherein at least one autonomic or neurologic index value as described above is considered to suggest whether a medical event is occurring (e.g., whether a POMEDA or a NOMEDA signal is present). For example, the autonomic or neurologic index value may be heart rate, and a heart rate value of at least 100 BPM may be taken as suggestive that an epileptic medical event is occurring. Generally, one or more values of one or more indices can be taken as suggestive that a medical event is occurring. If the autonomic or neurologic index value suggests a medical event is occurring, flow may be returned to the step subsequent to the “yes” decision, e.g., administering 640 or any one or more of the determining steps 681-690. If the autonomic or neurologic index value suggests a medical event is not occurring, flow may be directed to the continue monitoring step 630.

FIG. 6D depicts in more detail one particular embodiment of administering 640. In the embodiment depicted in FIG. 6D, a test is initially selected 641 based on one or more of the patient's baseline, the time of day, week, month, or year, values of one or more autonomic indices, or two or more thereof, among others.

The initially selected test may be modified 642 in view of prior test results or based on an ongoing event. “Modified” is used herein to mean the initially selected test may be made more difficult, less difficult, longer, shorter, replaced with a different cognitive test, or two or more thereof. For example, modifying may encompass increasing the volume of an auditory test, switching from a visual to a tactile or auditory test, or switching from a more complex cognitive test to a simpler test, among others.

The prior test results may be one or more of historical results over days, weeks, months, or even longer, or the results of previous iterations of the method administered during the same presumptive event.

The person of ordinary skill in the art will understand that variations in any of the depicted methods may be performed. For example, embodiments shown in FIGS. 6A-6C may be implemented together. For one example, the embodiment of FIG. 6C may be modified such that steps 610-660 are used to override a POMEDA determined from an autonomic or neurologic index value. For another example, the embodiments of FIGS. 6A-6C may be implemented at different times as part of the treatment regimen of one patient. For example, at different times of day, of the week, of the month, or of the year, the clinician may find it appropriate to perform different methods depicted in FIGS. 6A-6C.

In one embodiment, shown in FIG. 6E, the triggering event is a change in an autonomic or neurologic index, such as those discussed above. Upon a “yes” decision in node 675, flow returns to administering 640. Thereafter, it is determined at node 690 whether the patient's responsiveness is or is not impaired. If the patient's responsiveness is not impaired, a determination 691 is made whether there is an indication of a simple partial seizure. In one embodiment of the determination 691, the subject or an observer is asked if there is/was a clinical manifestation of a seizure, such as a sensation (typical for the seizure) or a visible manifestation. If yes, the seizure identified by node 675 is classified 692 as a simple partial seizure. If no, the seizure identified by node 675 is classified 693 as a subclinical seizure.

If the patient's responsiveness is impaired, a determination 694 is made whether a seizure severity value has exceeded a threshold. If yes, the seizure identified by node 675 is classified 695 as a generalized seizure. If no, the seizure identified by node 675 is classified 696 as a complex partial seizure.

As should be apparent from the foregoing discussion, the various method steps may be performed by one or more devices, such as a medical device 200 in concert with an external unit 270.

### EXAMPLE

A study was conducted at a major university medical center in subjects with pharmaco-resistant localization-related epilepsies undergoing surgical evaluation. After signing the consent form, subjects were enrolled into this study in the order of admission. The surgical assessment was conducted in accordance with this institution's protocol which included discontinuation of anti-seizure drugs or reductions in dose.

Inclusion criteria were: 1. Good candidate for invasive epilepsy surgery evaluation (subjects with at least one seizure/month on two or more appropriate medications at therapeutic serum concentrations); 2. Normal motor function; 3. Normal vision with or without correction; and 4. Low average IQ or higher.

Exclusion criteria were: 1. Mental retardation; 2. Status epilepticus during evaluation prior to collection of an adequate sample of test presentations and responses; 2. Use of rescue or psychoactive or CNS depressant drugs prior to collection of an adequate sample of test presentations and responses; 3. Medical or neurological complication prior to collection of an adequate sample of test presentations and responses and 4. Subject voluntary withdrawal prior to collection of an adequate sample of test presentations and responses.

Patient information is shown below in Tables 1-2.

To estimate the latency/time to impairment of complex reaction time responses from the time of electrographic onset recorded using depth or subdural electrodes, this test was administered to each subject under two conditions: a) Randomly and b) During seizures (triggered by automated detection). Complex reaction time tests were chosen in the expectation they would provide more insight into cognitive status than simple reaction time tests, while allowing frequent re-testing (without prominent training effect), as needed for this task, given the short average duration of seizures and of the study. Testing began no earlier than 24 hr after electrode implantation to allow for recovery from anesthesia and immediate postoperative pain/discomfort.

Test Description

Each subject received instructions as to how to take the test and had a training session that had to be succesfully completed prior to the start of the trial. The complex reaction time

(FIG. 7) consisted of the serial and simultaneous presentation of a pair of visual “stimuli” (the letter “A” and a square “□”), displayed full screen simultaneously on a 15″ monitor positioned at eye level, at a comfortable distance from the subject. The position of the letter on either half of the screen (i.e., left “A □”, or right “□A”), was randomly chosen for each presentation and the subject was instructed to immediately press, upon appearance of each visual stimuli, either the left or right mouse button according to the side of the screen (left or right) on which the letter A appeared. As soon as the subject pressed either button, or after a maximal presentation time (1 s) had elapsed, in case of no response, each stimulus presentation was removed (resulting in a blank screen) until the next stimuli. At the end of each presentation, a random timer was set, the expiration of which would trigger the next presentation. A total of 36 stimuli were presented in each testing session. Inter-trial presentation time intervals were randomly chosen from the set {0.5, 1.0, 1.5, 2.0 s.} to minimize adaptation and better assess performance.

Timing of Complex Reaction Time Tests Administration

The Complex Reaction-time tests were triggered only between 08:00-20:00 daily, throughout the surgical monitoring period by: a) Seizures [via the earlier of real-time automated seizure detection (Osorio et al, 2002) or event button presses] and b) Randomly. The timer that triggered random complex reaction time tests was set for 6 presentations per day, uniformly distributed throughout the 12 hour test period, with the additional constraints that no random test could occur within the 15 minutes period after a seizure or a randomly triggered test. To minimize fatigue, no more than a total of 30 tests (random plus seizure-triggered) could be administered over any 12 hour period.

Whenever a complex reaction time test was triggered, a sound file consisting of a voice saying “Begin Test” was automatically played to summon the subject to take the test. The subject was instructed to, upon hearing the summon, press one of the mouse buttons to activate the test. The “summons” was repeated every 5 s unless the subject began the test, up to a total of 6 times (30 s), with the sound file volume increasing with each repetition. If the subject did not initiate the complex reaction time test after 30 s, this information was logged and the system would go dormant until the next test.

Seizures were detected and quantified (intensity, duration and extent of spread) with a validated algorithm (Osorio et al 2002), whose output was used to trigger the complex reaction time tests; most automated detections occurred within 5 s of electrographic onset as marked visually by independent experts (Osorio et al, 2002). The classification of complex reaction time tests as ictal or random/interictal was validated off-line via expert visual analysis of the ECoG segments associated with each test. Complex reaction time tests triggered by false positive detections and randomly-triggered tests that overlapped in time with true seizures not detected by the algorithm (false negatives) were reclassified accordingly.

Complex Reaction Time Data Recording and Processing

The following were recorded (with millisecond precision) and logged/saved to the computer's memory: a) Test condition (random vs. seizure); b) Summon times corresponding to each prompt to the subject to start the test; c) Latency of responses to summons; d) Stimuli presentation times and side of the screen (left vs. right) where the letter “A” was displayed; and e) Times and sides (left vs. right) of all button presses. These data were processed to derive the following measures for each subject: I. Compliance, defined as the fraction of presented stimuli within each testing session for which the subject pressed the button regardless of correctness (Compliance Score=# responses/# presentations). II. Percentage of correct responses=# correct responses/# presentations, where correct responses are defined as those for which the subject pressed only once the button ipsilateral to the side (left or right) of the screen where the letter “A” appeared. Responses were classified as incorrect if: a) The mouse button contralateral to where the letter “A” was displayed on the screen was pressed; b) The right and left mouse buttons were pressed simultaneously or sequentially; c) The correct button was pressed more than once per stimulus presentation; or d) No button was pressed; III. Time to impaired response (TIR): The time (in seconds) elapsed between each test summon and the last correct response prior to the first test failure as defined below. The mean, range and standard deviation (SD) of time to impaired response (TIR) was computed for three different definitions of test failure, from most to least stringent: A) A correct response but with latency exceeding the 90^{th} percentile of those for random tests (TIR-A); speed of reaction is in certain situations as important as correctness of response; B) Any incorrect response as defined in II. Above (TIR-B); C) Three consecutive incorrect responses, regardless of their response latencies (TIR-C) , a definition that attempts to account for the fact that subjects make intermittent errors even during random tests when they presumably are not cognitively impaired. If no failure occurred in a test, the interval ends with the time of the correct response to the last stimuli. However, since assessment is limited to the duration (75 sec) of the complex reaction time test, the possibility of impairment after the test's termination cannot be excluded. Using the time to last correct response, not to the time to first failure (as defined above), overestimates time to impaired response (TIR), an approach deemed preferable/“safer” to underestimating it.

Subjects' data were included in the analyses only if: 1. The ECoG tracings were of sufficiently good quality to allow visual ascertainment of the presence or absence of seizures; and 2. There were at least two CRTs taken during random and at least one under seizure conditions.

ECoG Recording Processing and Analysis

ECoG was recorded using commercially available depth (mesial temporal regions) or grid/strip electrodes (cerebral convexities) electrodes (Ad-Tech, Racine, Wis.). These signals were fed into commercially available systems (Nicolet, Madison, Wis.), filtered (0.5-70 Hz; digitized (240 Hz, 10 bits of precision, 0.59 μV/bit) and further processed using a validated seizure detection and quantification algorithm (Osorio 1998, Osorio 2002) implemented into a custom bedside system (Peters et al., 2001). The detection algorithm quantified maximal seizure intensity (Si), duration (Sd); site(s) of seizure origin and extent of spread (Sc) were determined through visual review of ECoG. For this study, seizures were defined asany automated detection that reached an intensity threshold, T=22, for a minimum duration, D =0.84 s. with or without clinical manifestations. These parameters (T and D) were selected (Osorio et al, 98) to optimize sensitivity and specificity of the detection algorithm.

The relation between location and extent of the primary epileptogenic zone(s) and extent of seizure spread (outside the primary epileptogenic zone) and several complex reaction time performance measures (i.e. percent of correct responses; TIR, etc.) was probed. Seizure onset and spread were classified as follows: Focal: Ictal activity restricted to 2 contiguous electrode contacts; Regional: Ictal activity in 3 or more contiguous or non-contiguous contacts, provided the contacts are in the same region (i.e. left amygdala, pes and body of hippocampus); Lobar: Ictal activity in two or more regions within the same lobe (i.e., mesial temporal and neocortical temporal); Intrahemispheric: Ictal activity in two or more regions (in at least 2 different lobes) within the same hemisphere; Interhemispheric: Ictal activity in one or more regions (i.e. right and left mesial temporal regions) and Diffuse: Ictal activity in one or more lobes in each hemisphere.

In order to better understand the impact of seizure intensity (Si), duration (Sd) and extent of spread (Sc) on complex reaction time performance, the percentiles (p) of these three variables were conflated into one: Seizure Severity (SS)=pSi+pSd+pSc/3 (Osorio et al 2005) with one modification compared to the original one: using the classification defined above the following arbitrary values were assigned to it: Focal=1; Regional=2; Lobar=4; Intrahemispheric=8 and Interhemispheric=16. A scatter plot of seizure severity vs. percentage of correct response and time to impaired response (TIR) for each subject was generated and reviewed to assess the relationship between seizure severity and time to loss of function.

Data Processing Analyses

The data were analyzed for each as well as for all (pooled) subjects, where appropriate. Compliance, defined as the fraction of complex reaction time tests presented divided by those that were taken, was analyzed individually (as opposed to pooling the data from all subjects) since the data was skewed by one subject who was presented with 20 random tests and took 5 (25%) vs. 134 seizure tests of which 19 were taken (14%).

To provide additional insight and test for differences that might not be encompassed in an analysis of mean and SD alone, we analyzed the distributions of the various measures, comparing differences of random vs. seizure tests with the Kolmogorov-Smirnov test, a goodness of fit non-parametric test (Lindgren 1976). For each subject, the null hypothesis, namely, that the random and seizure tests performance are derived from the same distribution (i.e. they are not significantly (p=<0.05) different) was tested.

Since the deleterious impact of complex or secondarily generalized seizures on cognitive performance is cumulative, when closely spaced in time, all automated detections in a 15 minute window prior to each random or seizure test were annotated and taken into account in the interpretation of the complex reaction time test performance.

Results

Twenty subjects (See Table 1 for demographics, type, numbers and sites of electrode implantations and localization of the epileptogenic zone(s) all of which met the inclusion criteria, were enrolled in this study. The data from 6 subjects (4, 6, 7, 9, 12, 13) were excluded from analyses as they did not take the minimum required number of seizure-triggered tests. In 12/14 subjects electrographic onset preceded clinical seizure onset; in subjects 16 & 20 clinical preceded electrographic onset and their data was included in the analyses as control.

A total of 856 tests were administered: 649 (76%) were random with subjects responding to 520 (80%) and 207 (19%) were seizure-triggered (all true positive detections) with subjects responding to 73 (35%). These differences were primarily due to a few subjects who reported “getting tired” of taking large numbers of tests. The mean and SD of the average compliance scores for the 14 subjects included in the analyses were: Random tests: 0.91+/−0.12 vs. Seizure tests: 0.82+/−0.26, differences that were not statistically significant (paired t-test: p˜0.14). The mean and SD of the average percentage of correct responses for the 14 subjects were: Random tests: 85+/−14% vs. Seizure tests: 76+/−30%, differences that were not statistically significant (paired t-test: p˜0.15).

Mean maximal seizure intensity, duration and spread for each subject are shown in Table 3.

The mean, range and SD of time to failure (as defined above) for all subjects are shown in Table 4. Differences in means between seizure and random tests were significant for TIR-A (p˜0.02) and TIR-B (p˜0.04), but not for TIR-C (p˜0.4).

For 3/14 subjects, the Kolmogorov-Smirnov test identified significant differences in certain distributions: In subject 5, TIR-A was longer (p˜0.03) for seizures than for random tests; in subjects 8 & 11, the percentage of correct responses was higher (p˜0.04) for seizures than for random tests; in subject 11, the SD of response times for seizures is larger (p˜0.04) than for random tests.

The relationship between response delay and correctness of response to instantaneous seizure intensity (regardless of whether tests were triggered by seizure detection or by a random trigger; in the latter case, instantaneous seizure intensity was 0) was analyzed using scatter plots and classified into four groups: 1. As seizure intensity increased, response latency decreased, but without apparent impact on likelihood of correctness [Subject 14]; 2. As seizure intensity increased, response delay appeared unchanged, but with an increase in likelihood of an incorrect choice [Subjects 5, 16, and 20]; 3. As seizure intensity increased, response delay increased along with an increase in likelihood of an incorrect choice [Subjects 10 and 11]; and 4. As seizure intensity increased, there was no apparent change in either response delay or likelihood of an incorrect choice [Subjects 2, 17, and 19]. In the remaining five subjects, there was insufficient data at high intensities to identify any relationship between the variables [Subjects 1, 3, 8, 15, and 18].

Relative Seizure Severity (RSS) showed negative correlations between % correct and TIR-A,B,C with RSS for patients 2 (TIR-A), 3 (TIR-A,B), 4 (TIR-A,B,C), 5 (TIR-A,B), 6 (%corr, TIR-A,B ,C), 7. %corr, TIR-A,B,C), 9 (TIR-A,B), 11 (TIR-A,B), 12 (TIR-A,B), and 13 (TIR-B).

Discussion

This study employed automated seizure detection to trigger a complex reaction time test to estimate the length of time following automated seizure detection for which a subjects' performance is indistinguishable from interictal (non-seizure) periods. Reaction time is the time required for perceptual processing, evaluation of a stimulus and enactment of a response. Complex (also known as choice or alternative) reaction time, unlike simple reaction time tests, consists of more than one stimulus, adding complexity that increases with the logarithm of the stimuli number, thus probing thoroughly and in-depth a subject's ability to correctly and in a timely manner process and evaluate stimuli and generate an adaptive response. The process that takes place between the presentation of a stimulus and the response may be broken down into three subprocesses listed in order of occurrence: a) a stimulus registration time; b) a choice reaction time; and c) a time in constructing a decision to respond. This and the ability to re-administer it multiple times make complex reaction time tests suitable for assessing the impact of seizures on responsiveness/awareness.

The period (regardless of duration) after detection of seizure onset during which performance assessed with this complex reaction time test was indistinguishable from that obtained interictally, is referred herein to as Time to impaired response (TIR). It is inferred from these results that before impairment, subjects are able to acquire and correctly process sensory cues and integrate the elements required to generate an adaptive (appropriate) and timely response. The Time to Impaired Response obtained in this study, under adverse conditions (postoperatively and in an ICU environment), show that in subjects with seizures of mesial temporal origin (which are the majority in this cohort), the mean time to impaired response (TIR-A: 56.1 s; TIR-B: 27.1 s, and TIR-C: 42.8 s) was adequate for implementation/execution of certain behaviors, including but not limited to prevention of falls to the ground, other injuries, and even possibly disengagement from the operation of power equipment and of motor vehicles (Green, 2000). These findings justify the issuing of automated warning(s) (in addition to therapy) to decrease the risk of injuries and costs of care (both direct and indirect) and enhance the quality of life of subjects with seizures originating from certain brain regions. Due to the proclivity of seizures originating in the frontal lobe to rapidly evolve into complex or secondarily generalized ones and the susceptibility to dysfunction (even to single epileptiform discharges (Shewmon & Erwin I, II, 1988) of areas subserving vision, subjects with these epilepsies may not benefit from short-term warning.

That administration of the complex reaction time tests may have modified the probability of seizure occurrence, their expression, and severity is worth entertaining in light of two observations: 1. When compared with the 37 other subjects evaluated for epilepsy surgery during the same time period who were not enrolled in this study, those participating required significantly longer monitoring [by 2.2 days, 8.9 days for enrollees vs. 6.7 days for those not enrolled (p˜0.04)] to capture 5 typical clinical seizures; 2. In one subject (#2) the intensity of seizures during which tests were administered was significantly lower (p<0.001) than those during which tests were not administered. A small body of literature (Efron 1957; Paulson 1963; Kuhlman 1978; Papini et al 1984; Pritchard et al, 1985; Fenwick 1991) provides examples of seizure abatement, using sensory or other forms of stimulation, and of the increased likelihood of seizures with decreased vigilance and cognitive activity, suggesting that seizures may be amenable to “cognitive” intervention.

As gleaned from these observations, the systematic study of certain aspects of behavior and cognition during the peri-ictus and ictus illuminated heretofore unknown aspects of the mind-seizure interactions, provided means to decrease the burden of epileptics and of their caregivers, and expanded the realm of activities safely open to epileptics.

### REFERENCES

Block A, Fisher R S. Can patients perform volitional motor acts at the start of a seizure? J Clin Neurophysiol. 1999 March; 16(2):141-5.
Götz-Trabert K, Hauck C, Wagner K, Fauser S, Schulze-Bonhage A. Spread of ictal activity in focal epilepsy. Epilepsia. 2008 September; 49(9):1594-601. Epub 2008 Apr. 24.
Quesney L F.Clinical and EEG features of complex partial seizures of temporal lobe origin. Epilepsia. 1986;27 Suppl 2:S27-45.Links
Zajdel R, Nowak D. Simple and complex reaction time measurement. Computers in Biology and Medicine 37 (12); 1724-30 2007
Shewmon D A, Erwin R J.The effect of focal interictal spikes on perception and reaction time. I. General considerations. Electroencephalogr Clin Neurophysiol. 1988 April; 69(4):319-37. Shewmon D A, Erwin R J. The effect of focal interictal spikes on perception and reaction time. II. Neuroanatomic specificity. Electroencephalogr Clin Neurophysiol. 1988 April; 69(4):338-52.
Stern Y, Mayeux R, Cote L. Reaction time and vigilance in Parkinson's disease. Possible role of altered norepinephrine metabolism. Arch Neurol. 1984 October; 41(10):1086-9. Arnsten AF, Li BM. Neurobiology of executive functions: catecholamine influences on prefrontal cortical functions. Biol Psychiatry. 2005 Jun. 1; 57(11):1377-84.
Li G Y, Ueki H, Kawashima T, Sugataka K, Muraoka T, Yamada S. Involvement of the noradrenergic system in performance on a continuous task requiring effortful attention. Neuropsychobiology. 2004; 50(4):336-40
Egelman D M, Person C, Montague PR. A computational role for dopamine delivery in human decision-making. J Cogn Neurosci. 1998 September; 10(5):623-30.
Berridge C W, Waterhouse B D. The locus coeruleus-noradrenergic system: modulation of behavioral state and state-dependent cognitive processes. Brain Res Brain Res Rev. 2003 April; 42(1):33-84.
Shouse M N, Staba R J, Ko P Y, Saquib S F, Farber P R. Monoamines and seizures: microdialysis findings in locus ceruleus and amygdala before and during amygdala kindling. Brain Res. 2001 Feb. 16; 892(1):176-92.
Hoshino O. Cognitive enhancement mediated through postsynaptic actions of norepinephrine on ongoing cortical activity. Neural Comput. 2005 August; 17(8):1739-75.
Giorgi F S, Blandini F, Cantafora E, Biagioni F, Armentero M T, Pasquali L, Orzi F, Murri L, Paparelli A, Fornai F. Activation of brain metabolism and fos during limbic seizures: the role of locus coeruleus. Neurobiol Dis. 2008 June; 30(3):388-99. Epub 2008 Mar. 6.
Rammsayer T. Is there a common dopaminergic basis of time perception and reaction time? Neuropsychobiology 1989; 21:37-42
Aston-Jones G, Chiang C, Alexinsky T. Discharge of noradrenergic locus coeruleus neurons in behaving rats and monkeys suggests a role in vigilance. Prog Brain Res. 1991; 88:501-20.
Aston-Jones G, Chiang C, Alexinsky T. Discharge of noradrenergic locus coeruleus neurons in behaving rats and monkeys suggests a role in vigilance. Prog Brain Res. 1991; 88:501-20.
Fenwick P. Evocation and inhibition of seizures. Behavioral treatment. Adv Neurol. 1991; 55:163-83
Kuhlman W N. EEG feedback training of epileptic patients: clinical and electroencephalographic analysis. Electroencephalogr Clin Neurophysiol. 1978 December; 45(6):699-710.
Pritchard P B, Holmstrom V L, Giacinto J. Self-abatement of complex partial seizures. Ann Neurol 1985; 18:265-267.
Papini M, Pasquinelli A, Armellini M, Orlandi D. Alertness and incidence of seizures in patients with Lennox-Gastaut syndrome. Epilepsia 1984; 52:161-167.
Paulson G W. Inhibition of seizures. Dis Nery Syst 1963; 11:657-664.
Efron R. The conditioned inhibition of uncinate fits. Brain 1957; 80:251-262.
Lindgren B W. Statistical theory. 3^{rd} ed. New York: Macmillan, 1976:494-5.
Peters T E, Bhavaraju N C, Frei M G, Osorio I., J Clin Neurophysiol. 2001 November; 18(6):545-9.
Osorio I, Frei MG, Giftakis J, Peters T, Ingram J, Turnbull M, Herzog M, Rise M T, Schaffner S, Wennberg R A, Walczak T S, Risinger M W, Ajmone-Marsan C. Epilepsia. 2002 December; 43(12):1522-35.
Osorio I, Frei M G, Sunderam S, Giftakis J, Bhavaraju N C, Schaffner S F, Wilkinson S B. Ann Neurol. 2005 February; 57(2):258-68.
Osorio I, Frei M G, Wilkinson S B. Epilepsia. 1998 June; 39(6):615-27.
Green M. How Long Does It Take To Stop?' Methodological Analysis of Driver Perception-Brake Times. Transportation Human Factors, 2000; 2: 195-216.Begley, C. E., M. Famulari,
J. F. Annegers, D. R. Lairson, T. F. Reynolds, S. Coan, S. Dubinsky, M. E. Newmark, C. Leibson, E. L. So and W. A. Rocca (2000). “The cost of epilepsy in the United States: an estimate from population-based clinical and survey data.” Epilepsia 41(3): 342-51.
Kwan, P. and M. J. Brodie (2000). “Early identification of refractory epilepsy.” New England Journal of Medicine 342(5): 314-9.

TABLE 1

| Subject | Site(s) of Origin | # of SZs | Spread |
|---|---|---|---|
| 01 | RMT focal | 1 | No |
| 02 | LMT focal | 19 | Regional |
| 03 | RTnC | 2 | Focal |
| | RTnC regional | | No |
| 05 | RMT focal | 2 | Regional |
| 08 | LMT focal | 111 | No |
| | LMT regional | 8 | No |
| | LMT focal | 2 | Interhemispheric |
| 10 | LMT regional | 7 | No |
| 11 | LMT regional | 2 | Intrahemispheric |
| 14 | Interhemispheric | 10 | No |
| 15 | R frontal regional | 9 | No |
| | R frontal regional | 1 | Lobar |
| 16 | L frontal focal | 1 | Diffuse |
| 17 | RMT focal | 1 | Regional |
| | RMT focal | 1 | Interhemispheric |
| | RMT focal | 1 | No |
| | RMT focal | 1 | Regional |
| | LMT focal | 1 | Interhemispheric |
| | RTM focal | 1 | No |
| 18 | LMT focal | 4 | Regional |
| | LMT focal | 1 | No |
| 19 | RTM focal | 1 | No |
| | RTM focal | 1 | Regional |
| 20 | RTM Regional | 1 | Interhemispheric |

L = Left;
R = Right;
M = Mesial;
T = Temporal;
nC = Neocortical

TABLE 2

| | | Years | | | | Monthly | Primary | Electrode |
|---|---|---|---|---|---|---|---|---|
| Subject | | with | | | | Baseline | Epileptogenic | Type(s) & |
| No. | Age | epilepsy | Gender | Etiology | Class | Frequency | Zone(s) | Approach |
| 1 | 32 | 27 | F | Cryptogenic | CP | 10 | Rt. Amygdala | B.D.; Lat. |
| 2 | 30 | 29 | F | Cryptogenic | CP | 4 | Lt. Amygdala & Pes | B.D.; Lat. |
| | | | | | | | Hippocampus | |
| 3 | 34 | 22 | M | Infection | Sec. | 5 | Rt. Fronto-Temporal | Grid |
| | | | | | Gral. | | Convexity | |
| 4 | 22 | 4 | M | Trauma | Sec. | 5 | Rt. Hippocampus | B.D.; Lat. |
| | | | | | Gral. | | | |
| 5 | 11 | 4 | M | Cryptogenic | CP | 16 | Rt. Amygdala | B.D.; Lat. |
| 6 | 30 | 23 | F | Congenital | CP | 10 | Rt. Amygdala & | B.D.; Lat. |
| | | | | | | | Hippocampus | |
| 7 | 42 | 14 | M | Trauma | Sec. | 7 | Rt. Frontal | Grid |
| | | | | | Gral. | | Convexity | |
| 8 | 23 | 23 | F | Cryptogenic | CP | N/A | Lt. Hippocampus | B.D.; Lat. |
| 9 | 35 | 34 | F | Trauma | Sec. | 4 | Lt. Frontal | Grid |
| | | | | | Gral. | | Convexity | |
| 10 | 26 | 26 | M | Infection | CP | 15 | Left Amygdala & | B.D.; Lat. |
| | | | | | | | Hippocampus | |
| 11 | 48 | 38 | M | Cryptogenic | CP | 75 | Rt Amygdala | B.D.; Lat. |
| 12 | 20 | 8 | M | Cryptogenic | C P | 2 | Rt. Posterior | B.D.; Lai |
| | | | | | | | Hippocampus | |
| 13 | 28 | 3 | M | Cryptogenic | CP | 45 | Bi-occipital polar | B.D. |
| 14 | 34 | 22 | F | Cryptogenic | CP | 3 | Lt. Hippocampus; Rt. | B.D.; Lat. |
| | | | | | | | Amygdala | |
| 15 | 32 | 19 | M | Trauma | Sec. | 3 | Rt. Frontal Convexity | Grid |
| | | | | | Gral. | | | |
| 16 | 22 | 21 | F | Cryptogenic | Sec. | 2 | Rt. Post. | B.D.; Str |
| | | | | | Gral. | | Hippocampus; Lt. | |
| | | | | | | | Post. Hippocampus; | |
| | | | | | | | bi- | |
| | | | | | | | Fronto-Polar | |
| 17 | 19 | 11 | M | Cryptogenic | CP | 3 | Lt. Hippocampus; Rt. | B.D.; Lat. |
| | | | | | | | Amygdala | |
| 18 | 21 | 17 | M | Trauma | CP | 4 | Lt. Hippocampus | B.D.; Lat. |
| 19 | 25 | 16 | F | Cryptogenic | Sec. | N/A | Lt. Hippocampus | B.D.; Lat. |
| | | | | | Gral. | | | |
| 20 | 30 | 11 | M | Cryptogenic | CP | 16 | Rt. Amygdala & | B.D.; Lat. |
| | | | | | | | Hippocampus | |

CP = Complex Partial; Sec. Gral. = Secondarily Generalized; B.D. = BilateralDepth electrodes; Lat. = Lateral (Electrodes are inserted through the temporal bone)

TABLE 3

| Subject | Mean Maximal | Mean | Mean |
|---|---|---|---|
| Seizures | Intensity | Duration (s) | Spread |
| 01 | 17.7 | 4.0 | 1.0 |
| 02 | 124.8 | 28.9 | 2.0 |
| 03 | 5.5 | 34.0 | 1.5 |
| 05 | 306.5 | 17.1 | 2.0 |
| 08 | 94.6 | 6.4 | 1.3 |
| 10 | 247.0 | 22.6 | 2.0 |
| 11 | 607.1 | 79.4 | 8.0 |
| 14 | 128.8 | 16.7 | 16.0 |
| 15 | 165.3 | 4.9 | 2.2 |
| 16 | 1005.7 | 36.2 | 16.0 |
| 17 | 242.0 | 9.6 | 6.3 |
| 18 | 842.6 | 25.8 | 5.6 |
| 19 | 3.6 | 70.0 | 1.5 |
| 20 | 340.3 | 87.6 | 16.0 |

TABLE 4

| | Random Tests | | | Seizure Tests | | |
|---|---|---|---|---|---|---|
| | Mean | SD | Range | Mean | SD | Range |
| | (s) | (s) | (s) | (s) | (s) | (s) |
| TIR-A | 22.0 | 17.3 | 0.2-83.5 | 27.1 | 19.8 | 0.6-75.0 |
| TIR-B | 37.2 | 25.0 | 0.2-92.0 | 42.8 | 24.2 | 0.6-82.2 |
| TIR-C | 55.5 | 24.8 | 2.0-103.0 | 56.1 | 23.9 | 0.6-88.7 |

TIR: Time to impaired response, with failure defined as: A. Either an incorrect response or a slow correct response (with latency exceeding the 90%tile of random test response latencies); B. Any incorrect response C. Three consecutive incorrect responses

All of the methods and apparatuses disclosed and claimed herein may be made and executed without undue experimentation in light of the present disclosure. While the methods and apparatus of this invention have been described in terms of particular embodiments, it will be apparent to those skilled in the art that variations may be applied to the methods and apparatus and in the steps, or in the sequence of steps, of the method described herein without departing from the concept, spirit, and scope of the invention, as defined by the appended claims. It should be especially apparent that the principles of the invention may be applied to selected cranial nerves other than, or in addition to, the vagus nerve to achieve particular results in treating patients having epilepsy, depression, or other medical conditions.

The particular embodiments disclosed above are illustrative only as the invention may be modified and practiced in different but equivalent manners apparent to those skilled in the art having the benefit of the teachings herein. Furthermore, no limitations are intended to the details of construction or design herein shown other than as described in the claims below. It is, therefore, evident that the particular embodiments disclosed above may be altered or modified and all such variations are considered within the scope and spirit of the invention. Accordingly, the protection sought herein is as set forth in the claims below.

What is claimed:

1. A method for determining a degree of responsiveness of a patient having brain state changes, comprising:
receiving an indication of a triggering event;
administering to the patient, in response to the indication, a test of responsiveness; and
determining, based upon a result of the test, at least one responsiveness parameter selected from the group consisting of (i) a time of occurrence of a change in the patient's responsiveness, (ii) a duration of a change in the patient's responsiveness; (iii) a magnitude of a change in the patient's responsiveness, (iv) a time interval from the indication of event occurrence to a change in the patient's responsiveness, (v) a type of change in the patient's responsiveness, (vi) an estimation of a seizure severity; (vii) a classification of a seizure into clinical or subclinical; (viii) a classification of a clinical seizure into simple partial, complex partial, or generalized; (ix) an assessment of efficacy of a therapy for the patient's medical condition; (x) an assessment of the state of the disease and formulation of a prognosis for the patient; (xi) an estimation of a risk of injury or death for the patient; and (xii) two or more thereof.

2. The method of claim 1, wherein the triggering event is selected from the group consisting of a) an indication from a medical event detection algorithm that a medical event is occurring or is imminent; b) a manual signal to administer the responsiveness test to the patient; or c) a command to administer a responsiveness test to the patient in the absence of an indication from a medical event detection algorithm that a medical event is occurring or imminent.

3. The method of claim 1, further comprising detecting a change in an autonomic index of the patient by analyzing at least one set of signals received from the patient and selected from the group consisting of cardiovascular signals, respiratory signals, skin signals, pupillary signals, temperature signals, peristaltic signals, autonomic nerve or ganglia signals, and two or more thereof.

4. The method of claim 1, further comprising detecting a change in a neurologic index of the patient by analyzing at least one set of signals received from the patient and selected from the group consisting of brain signals, cranial nerve signals, spinal cord signals, peripheral nerve signals, body kinetic, position and force signals, and two or more thereof.

5. The method of claim 1, wherein the patient suffers from epilepsy, and administering is performed at a plurality of times, wherein at least one of the plurality of times is ictal and at least one of the plurality of times is nonictal.

6. The method of claim 1, further comprising selecting a first test of responsiveness having a first difficulty level, and, based on the patient's responsiveness according to the first test, selecting and administering a second test of responsiveness having a second difficulty level.

7. The method of claim 1, further comprising selecting a first test of responsiveness having a first duration, and, based on the patient's responsiveness according to the first test, selecting and administering a second test of responsiveness having a second duration.

8. The method of claim 1, wherein the test of responsiveness tests at least one of the patient's reflex, motor, or cognitive functions.

9. The method of claim 1, wherein the test of responsiveness tests a cognitive function of the patient, wherein the cognitive function is selected from the group consisting of attention, verbal, non-verbal and procedural short-term memory, verbal, non-verbal and procedural long-term memory, language fluency and comprehension, visuo-spatial functions, auditory discrimination, visual discrimination, abstract reasoning, calculations, or two or more thereof.

10. The method of claim 1, further comprising delivering a therapy for a seizure event to the patient, wherein the therapy for the seizure event is selected from the group consisting of electrical stimulation of a cranial nerve of the patient, thermal manipulation of the cranial nerve of the patient, electrical stimulation of the brain of the patient, thermal manipulation of the brain of the patient, delivery of a chemical agent to the patient via the bloodstream, cerebrospinal fluid or directly into the brain, magnetic stimulation of a cranial nerve, magnetic stimulation of the brain a motor task, a perceptual task, a cognitive task, and two or more thereof.

11. The method of claim 1, further comprising, based on the patient's responsiveness, instructing an external device to change an operating state thereof.

12. A computer readable program storage unit encoded with instructions that, when executed by a computer, perform a method for determining a responsiveness of a patient having brain state changes, comprising:
receiving an indication of a triggering event;
administering to the patient, in response to the indication, a test of responsiveness; and
determining, based upon a result of the test, at least one responsiveness parameter selected from the group consisting of (i) a time of occurrence of a change in the patient's responsiveness, (ii) a duration of a change in the patient's responsiveness; (iii) a magnitude of a change in the patient's responsiveness, (iv) a time interval from the indication of event occurrence to a change in the patient's responsiveness, (v) a type of change in the patient's responsiveness, (vi) an estimation of a seizure severity; (vii) a classification of a seizure into clinical or subclinical; (viii) a classification of a clinical seizure into simple partial, complex partial, or generalized; (ix) an assessment of efficacy of a therapy for the patient's medical condition; (x) an assessment of the state of the disease and formulation of a prognosis for the patient; (xi) an estimation of a risk of injury or death for the patient; and (xii) two or more thereof .

13. The computer readable program storage unit of claim 12, wherein the triggering event is selected from the group consisting of a) an indication from a medical event detection algorithm that a medical event is occurring or is imminent; b) a manual signal to administer the responsiveness test to the patient; or c) a command to administer a responsiveness test to the patient in the absence of an indication from a medical event detection algorithm that a medical event is occurring or imminent.

14. The computer readable program storage unit of claim 12, further comprising detecting a change in an autonomic index of the patient by analyzing at least one set of signals received from the patient and selected from the group consisting of cardiovascular signals, respiratory signals, skin signals, pupillary signals, temperature signals, peristaltic signals, autonomic nerve or ganglia signals, and two or more thereof

15. The computer readable program storage unit of claim 12, further comprising detecting a change in a neurologic index of the patient by analyzing at least one set of signals received from the patient and selected from the group consisting of brain signals, cranial nerve signals, spinal cord signals, peripheral nerve signals, body kinetic, position and force signals, and two or more thereof

16. The computer readable program storage unit of claim 12, wherein the patient suffers from epilepsy, and administering is performed at a plurality of times, wherein at least at least one of the plurality of times is ictal and at least one of the plurality of times is nonictal.

17. The computer readable program storage unit of claim 12, further comprising selecting a first test of responsiveness having a first difficulty level, and, based on the patient's responsiveness according to the first test, selecting and administering a second test of responsiveness having a second difficulty level.

18. The computer readable program storage unit of claim 12, further comprising selecting a first test of responsiveness having a first duration, and, based on the patient's responsiveness according to the first test, selecting and administering a second test of responsiveness having a second duration.

19. The computer readable program storage unit of claim 12, wherein the test of responsiveness tests at least one of the patient's reflex, motor, or cognitive functions.

20. The computer readable program storage unit of claim 12, wherein the test of responsiveness tests a cognitive function of the patient, wherein the cognitive function is selected from the group consisting of attention, verbal, non-verbal and procedural short-term memory, verbal, non-verbal and procedural long-term memory, language fluency and comprehension, visuo-spatial functions, auditory discrimination, visual discrimination, abstract reasoning, calculations, or two or more thereof.
